(19) United States
(12) Patent Application Publication
(10) Pub. No.: US 2011/0251247 A1
(43) Pub. Date: Oct. 13, 2011

(54) SUBSTITUTED ISOXAZOLINE DERIVATIVES

(75) Inventors: Nathan Anthony Logan Chubb, Richland, MI (US); Todd Maddux, Kalamazoo, MI (US); Sanjay Menon, Kalamazoo, MI (US)

(73) Assignee: PFIZER INC., New York, NY (US)

(21) Appl. No.: 13/080,721

(22) Filed: Apr. 6, 2011

Related U.S. Application Data
(60) Provisional application No. 61/322,144, filed on Apr. 8, 2010.
(60) Provisional application No. 61/431,107, filed on Jan. 10, 2011.

(51) Int. Cl. A61K 31/42; A61P 33/00; C07D 261/04

(52) U.S. Cl. 514/378; 548/240

(57) ABSTRACT

This invention recites substituted isoxazoline derivatives of Formula (1)

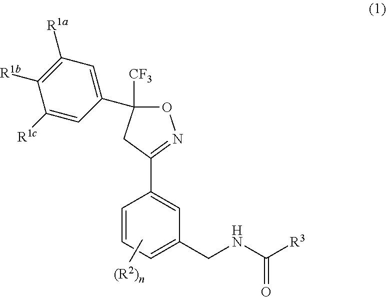

or a veterinarily acceptable salt thereof, with parasiticidal activity, compositions thereof, and their use as a parasiticide in animals or birds where R^{1a}, R^{1b}, R^{1c}, R², R³, and n are as described herein.

### CROSS-REFERENCE TO RELATED APPLICATIONS

This application claims benefit of priority from pending U.S. Provisional Application Ser. No. 61/322,144, filed Apr. 8, 2010 and U.S. Provisional Application Ser. No. 61/431,107, filed Jan. 10, 2011.

### FIELD OF THE INVENTION

This invention relates to isoxazoline derivatives having parasiticidal activity.

The compounds of interest are substituted isoxazoline 3-benzyl acetamides, carbamates, and ureas. The invention also relates to veterinary compositions and methods of use thereof.

### BACKGROUND

There is a need for improved antiparasitic agents for use with animals and birds, and in particular there is a need for improved insecticides and acaricides. Furthermore, there is a need for improved topical and oral products with convenient administration and which contain at least one of such antiparasitic agent which can be used to effectively treat ectoparasites, such as insects (e.g., fleas, lice, and flies) and acarids (e.g., mites and ticks). Such products would be particularly useful for the treatment of companion animals, livestock, and fowl.

The compounds currently available for insecticidal and acaricidal treatment of companion animals, livestock, and fowl do not always demonstrate good activity, speed of action, or duration of action. Most treatments contain hazardous chemicals that can have serious consequences, including lethality from accidental ingestion. Persons applying these agents are generally advised to limit their exposure. Pet collars and tags have been utilized to overcome some problems, but these are susceptible to chewing, ingestion, and subsequent toxicological affects to the animal. Thus, current treatments achieve varying degrees of success which depend partly on toxicity, method of administration, and efficacy. Currently, some agents are actually becoming ineffective due to parasitic resistance.

Isoxazoline derivatives have been disclosed in the art as having insecticidal and acaricidal activity. For example, WO2005/085216, WO2007/105814, WO2007/026965, WO2008/122375, and JP2008239611 describe 4-(5-substituted-5-substituted aryl-4,5-dihydroisoxazole-3-yl)benzamide and amine derivatives. Further, WO2005/051932 recites certain 4,5-dihydroisoxazole benzamide derivatives but does not disclose compounds of the instant invention. Despite the availability of effective, broad spectrum antiparasitic agents, there remains a need for a safer, convenient, and environmentally friendly product that will overcome the ever-present threat of resistance development.

These citations do not exemplify any isoxazoline substituted oxazoles of the present invention, nor do they indicate that such compounds would be useful against a spectrum of parasitic species relevant to companion animals, livestock, fowl, or against the range of parasitic morphological lifecycle stages.

The present invention overcomes one or more of the various disadvantages of, or improves upon, the properties of existing compounds. In particular the present invention describes new isoxazoline substituted aryl and heteroaryl oxazoles which demonstrate such properties.

### SUMMARY

The present invention provides Formula (1) compounds, or a veterinarily acceptable salt thereof, which act as parasiticides, in particular, ectoparasiticides; therefore may be used to prevent, treat, repel, and control acarids and insect infection and infestation in animals and birds. In addition, the invention contemplates the control and prevention of tick borne diseases, for example, Lyme disease, canine and bovine anaplasmosis, canine ehrlichiosis, canine rickettsiosis, canine and bovine babesiosis, epizootic bovine abortion, and theileriosis. Thus, according to the present invention, there is provided a compound of Formula (1)

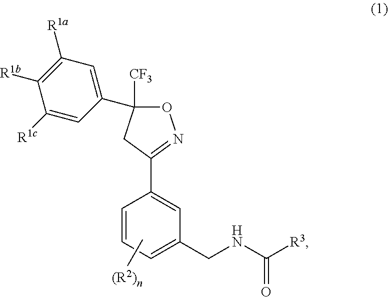

or a veterinarily acceptable salt thereof, wherein

R^{1a}, R^{1b}, and R^{1c} are each independently selected from halogen, cyano, C₁-C₈ alkyl, C₁-C₆ haloalkyl, and C₁-C₆ haloalkoxy, and each R¹ may be identical with or different from each other;

R² is hydrogen, halo, cyano, C₁-C₆ alkyl, C₁-C₆ haloalkyl, or C₁-C₆ alkoxy, C₁-C₆ haloalkoxy, C₃-C₆cycloalkyl, where n is an integer 1, 2, or 3, and when n is 2 or 3, each R² may be identical with or different from each other;

R³ is selected from C₁-C₈ alkyl, C₀-C₃ alkylC₃-C₆ cycloalkyl, C₁-C₆ alkyl-OR⁴, or C₁-C₆ alkylC(O)NR^{a}R^{b}, wherein the C₁-C₈ alkyl and the C₀-C₃ alkylC₃-C₆ cycloalkyl are optionally substituted with at least one substituent selected from halo, cyano, hydroxyl, and S(O)ₚR⁴;

R⁴ is C₁-C₆ alkyl or C₁-C₆ haloalkyl;

R^{a} is hydrogen or C₁-C₆ alkyl;

R^{b} is hydrogen, C₁-C₆ alkyl, C₁-C₆ haloalkyl, C₀-C₄alkylC₃-C₆cycloalkyl, or C₁-C₃alkylHet, wherein Het is a 5- or 6-membered monocyclic aromatic ring containing at least one heteroatom selected from N, O, or S, and the Het can be optionally substituted with at least one substituent selected from halo, cyano, C₁-C₆ alkyl, and C₁-C₆ haloalkyl; and

p is the integer 0, 1, or 2.

In another aspect of the invention (R²)ₙ is R^{2a}, R^{2b}, and R² when the integer n is 3. When the integer n is 2, then (R²)ₙ is R^{2a} and R^{2b}, R^{2a} and R^{2c}, or R^{2b} and R^{2c}. When the integer n is 1, then (R²)ₙ is R^{2a}, R^{2b}, or R^{2c}.

In another aspect of the invention, compounds of Formula (1) include compounds of Formula (1A), (1B), (1C), and (1D)

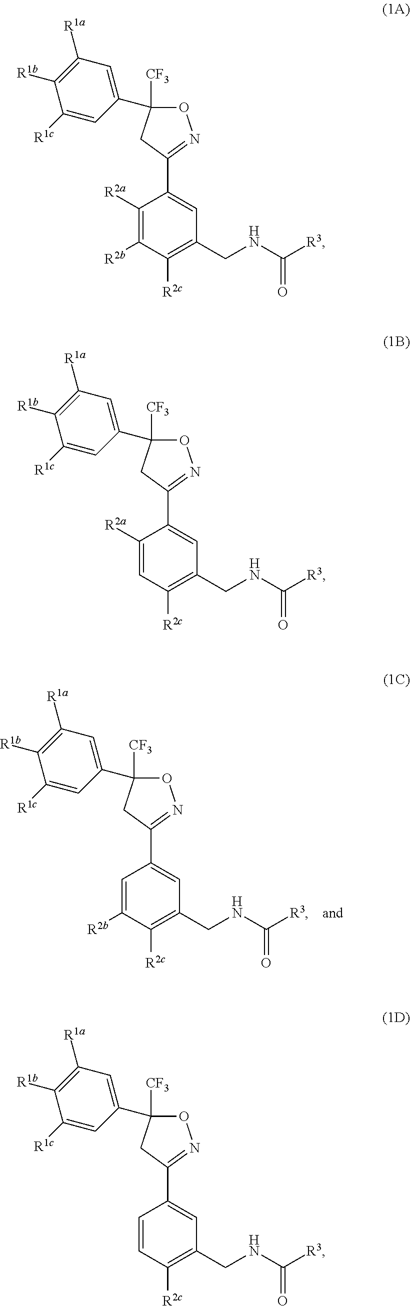

or a veterinarily acceptable salt thereof.

In another aspect of the invention, compounds of Formula (1) include compounds of Formula (1A). In yet another aspect of the invention, compounds of Formula (1) include compounds of Formula (1B). In yet another aspect of the invention, compounds of Formula (1) include compounds of Formula (1C). In yet another aspect of the invention, compounds of Formula (1) include compounds of Formula (1D).

In another aspect of the invention, R^{1a}, R^{1b}, and R^{1c} are each independently selected from halogen, cyano, C₁-C₈ alkyl, or C₁-C₆ haloalkyl. In yet another aspect of the invention, R^{1a}, R^{1b}, and R^{1c} are each independently selected from fluoro, chloro, bromo, cyano, C₁-C₈ alkyl, and C₁-C₆ haloalkyl. In yet another aspect of the invention, R^{1a}, R^{1b}, and R^{1c} are each independently selected from fluoro, chloro, bromo, cyano, methyl, ethyl, —CF₃, and —CH₂CF₃. In yet another aspect of the invention, R^{1a}, R^{1b}, and R^{1c} are each independently selected from fluoro, chloro, bromo, and CF₃. In still another aspect of the invention, R^{1a}, R^{1b}, and R^{1c} are each independently selected from fluoro or chloro. In still another aspect of the invention, R^{1a} and R^{1c} are each chloro and R^{1b} is fluoro. In still another aspect of the invention, R^{1a}, R^{1b}, and R^{1c} are each chloro.

In another aspect of the invention, R^{2a}, R^{2b}, and R^{2c} are each independently hydrogen, halo, cyano, C₁-C₆ alkyl, C₁-C₆ haloalkyl, or C₃-C₆ cycloalkyl. In yet another aspect of the invention, R^{2a}, R^{2b}, and R^{2c} are each independently hydrogen, halo, cyano, methyl, ethyl, —CF₃, —CH₂CF₃, cyclopropyl or cyclobutyl. In yet another aspect of the invention, R^{2a}, R^{2b}, and R^{2c} are each independently hydrogen, fluoro, chloro, bromo, cyano, methyl, or CF₃. In yet another aspect of the invention, R^{2a}, R^{2b}, and R^{2c} are each independently fluoro, chloro, bromo, methyl, or CF₃.

In yet another aspect of the invention, R^{2a} and R^{2b} are both hydrogen and R^{2c} is hydrogen, halo, cyano, methyl, ethyl, —CF₃, —CH₂CF₃, cyclopropyl or cyclobutyl. In yet another aspect of the invention, R^{2a} and R^{2b} are both hydrogen and R^{2c} is hydrogen, fluoro, chloro, bromo, cyano, methyl, or CF₃. In yet another aspect of the invention, R^{2a} and R^{2b} are both hydrogen and R^{2c} is fluoro, chloro, bromo, methyl, or CF₃. In yet another aspect of the invention, R^{2a} and R^{2b} are both hydrogen and R^{2c} is fluoro, chloro, or bromo. In yet another aspect of the invention, R^{2a} and R^{2b} are both hydrogen and R^{2c} is fluoro. In yet another aspect of the invention, R^{2a} and R^{2b} are both hydrogen and R^{2c} is chloro. In yet another aspect of the invention, R^{2a} and R^{2b} are both hydrogen and R^{2c} is bromo.

In yet another aspect of the invention, R³ is selected from C₁-C₈ alkyl or C₀-C₃alkylC₃-C₆ cycloalkyl; wherein the C₁-C₈ alkyl and the C₀-C₃ alkylC₃-C₆ cycloalkyl are optionally substituted with at least one substituent selected from halo, hydroxyl, and S(O)ₚR⁴ where p is the integer 0, 1, or 2, and R⁴ is methyl, ethyl, or isopropyl.

In yet another aspect of the invention, R³ is selected from C₁-C₈ alkyl, cyclopropyl, cyclobutyl, cyclopentyl, methylcyclopropyl, ethylcyclopropyl, methylcyclobutyl, ethylcyclobutyl, and methyl cyclopentyl; wherein the alkyl, cycloalkyl, and alkylcycloalkyl are optionally substituted with at least one substituent selected from halo, hydroxyl, —SCH₃, and —S(O)₂CH₃.

In yet still another aspect of the invention, R³ is selected from methyl, ethyl, propyl, butyl, isopropyl, isobutyl, n-butyl, t-butyl, cyclopropyl, cyclobutyl, cyclopentyl, methylcyclopropyl, ethylcyclopropyl, methylcyclobutyl, ethylcyclobutyl, and methyl cyclopentyl; wherein the alkyl, cycloalkyl, and the alkylcycloalkyl are optionally substituted with at least one substituent selected from halo, hydroxyl, —SCH₃, and —S(O)₂CH₃.

In yet still another aspect of the invention, R³ is selected from methyl, ethyl, propyl, butyl, isopropyl, isobutyl, n-butyl, t-butyl, cyclopropyl, cyclobutyl, cyclopentyl, methylcyclopropyl, ethylcyclopropyl, methylcyclobutyl, ethylcyclobutyl, and methyl cyclopentyl; wherein the alkyl, cycloalkyl, and the alkylcycloalkyl are optionally substituted with at least one substituent selected from fluoro, chloro, —SCH₃, and —S(O)₂CH₃.

In yet still another aspect of the invention, R³ is selected from methyl, ethyl, isopropyl, isobutyl, cyclopropyl, cyclobutyl, and methylcyclopropyl; wherein the alkyl, cycloalkyl, and the alkylcycloalkyl are optionally substituted with at least one substituent selected from fluoro, chloro, —SCH₃, and —S(O)₂CH₃. In yet still another aspect of the invention, R³ is selected from methyl, ethyl, isopropyl, isobutyl, cyclopropyl, cyclobutyl, and methylcyclopropyl.

In yet another aspect of the invention, R³ is C₁-C₆alkyl-OR⁴, where C₁-C₆alkyl is methyl, ethyl, or propyl, and R⁴ is methyl, ethyl, isopropyl, or trifluoromethyl. In yet another aspect of the invention, R³ is —CH₂—O—CH₃, —CH₂—O—CH₂CH₃, or —CH₂—O—CF₃.

In yet another aspect of the invention, R³ is C₁-C₆ alkylC(O)NR^{a}R^{b}, where C₁-C₆alkyl is methyl or ethyl, R^{a} is hydrogen and R^{b} is methyl, ethyl, trifluoromethyl, methylcyclopropyl, —CH₂-pyrazole, —CH₂-oxazole, —CH₂-imidazole, —CH₂-thiazolyl, —CH₂-isothiazolyl, —CH₂-triazole, —CH₂-tetrazole, —CH₂-pyridine, —CH₂-pyridazine, and —CH₂-pyrimidine. In yet another aspect of the invention R^{b} is methy, ethyl, methylcyclopropyl, —CH₂-pyrazole, —CH₂-imidazole, —CH₂-triazole, —CH₂-tetrazole, —CH₂-pyridine, —CH₂-pyridazine, and —CH₂-pyrimidine.

In yet another aspect of the invention, the integer p is 0. In yet another aspect of the invention, the integer p is 1. In yet another aspect of the invention, the integer p is 2.

In another aspect of the invention, Formula (1) compounds include:
N-{5-[5-(3,4,5-trichlorophenyl)-5-(trifluoromethyl)-4,5-dihydroisoxazol-3-yl]-2-fluorobenzyl}acetamide;
N-{2-fluoro-5-[5-(3,4,5-trichlorophenyl)-5-(trifluoromethyl)-4,5-dihydroisoxazol-3-yl]benzyl}-2-methylpropanamide;
N-{2-fluoro-5-[5-(3,4,5-trichlorophenyl)-5-(trifluoromethyl)-4,5-dihydroisoxazol-3-yl]benzyl}cyclopropanecarboxamide;
N-{2-fluoro-5-[5-(3,4,5-trichlorophenyl)-5-(trifluoromethyl)-4,5-dihydroisoxazol-3-yl]benzyl}cyclobutanecarboxamide;
N-{2-fluoro-5-[5-(3,4,5-trichlorophenyl)-5-(trifluoromethyl)-4,5-dihydroisoxazol-3-yl]benzyl}propanamide;
2-cyclopropyl-N-{2-fluoro-5-[5-(3,4,5-trichlorophenyl)-5-(trifluoromethyl)-4,5-dihydroisoxazol-3-yl]benzyl}acetamide;
N-{2-fluoro-5-[5-(3,4,5-trichlorophenyl)-5-(trifluoromethyl)-4,5-dihydroisoxazol-3-yl]benzyl}-3-methylbutanamide;
2-cyclopropyl-N-{5-[5-(3,5-dichloro-4-fluorophenyl)-5-(trifluoromethyl)-4,5-dihydroisoxazol-3-yl]-2-fluorobenzyl}acetamide;
N-{5-[5-(3,5-dichloro-4-fluorophenyl)-5-(trifluoromethyl)-4,5-dihydroisoxazol-3-yl]-2-fluorobenzyl}acetamide;
N-{5-[5-(3,5-dichloro-4-fluorophenyl)-5-(trifluoromethyl)-4,5-dihydroisoxazol-3-yl]-2-fluorobenzyl}cyclopropanecarboxamide;
N-{5-[5-(3,5-dichloro-4-fluorophenyl)-5-(trifluoromethyl)-4,5-dihydroisoxazol-3-yl]-2-fluorobenzyl}-3,3-difluorocyclobutanecarboxamide
N-{2-chloro-5-[5-(3,4,5-trichlorophenyl)-5-(trifluoromethyl)-4,5-dihydroisoxazol-3-yl]benzyl}acetamide;
N-{2-chloro-5-[5-(3,4,5-trichlorophenyl)-5-(trifluoromethyl)-4,5-dihydroisoxazol-3-yl]benzyl}cyclopropanecarboxamide;
N-{2-chloro-5-[5-(3,4,5-trichlorophenyl)-5-(trifluoromethyl)-4,5-dihydroisoxazol-3-yl]benzyl}-2-methylpropanamide;
N-{2-chloro-5-[5-(3,5-dichloro-4-fluorophenyl)-5-(trifluoromethyl)-4,5-dihydroisoxazol-3-yl]benzyl}acetamide;
N-(2-bromo-5-(5-(3,4,5-trichlorophenyl)-5-(trifluoromethyl)-4,5-dihydroisoxazol-3-yl)benzyl)acetamide,
N-Cyclopropylmethyl-N′-{2-fluoro-5-[5-(3,4,5-trichloro-phenyl)-5-trifluoromethyl-4,5-dihydro-isoxazol-3-yl]-benzyl}-malonamide; and
N-ethyl-N′-{2-fluoro-5-[5-(3,4,5-trichloro-phenyl)-5-trifluoromethyl-4,5-dihydro-isoxazol-3-yl]-benzyl}-malonamide, or a veterinarily acceptable salt thereof.

In yet another aspect of the invention, Formula (1) compounds include:
N-{5-[5-(3,4,5-trichlorophenyl)-5-(trifluoromethyl)-4,5-dihydroisoxazol-3-yl]-2-fluorobenzyl}acetamide;
N-{2-fluoro-5-[5-(3,4,5-trichlorophenyl)-5-(trifluoromethyl)-4,5-dihydroisoxazol-3-yl]benzyl}-2-methylpropanamide;
N-{2-fluoro-5-[5-(3,4,5-trichlorophenyl)-5-(trifluoromethyl)-4,5-dihydroisoxazol-3-yl]benzyl}cyclopropanecarboxamide;
N-{2-fluoro-5-[5-(3,4,5-trichlorophenyl)-5-(trifluoromethyl)-4,5-dihydroisoxazol-3-yl]benzyl}cyclobutanecarboxamide;
N-{2-fluoro-5-[5-(3,4,5-trichlorophenyl)-5-(trifluoromethyl)-4,5-dihydroisoxazol-3-yl]benzyl}propanamide;
2-cyclopropyl-N-{2-fluoro-5-[5-(3,4,5-trichlorophenyl)-5-(trifluoromethyl)-4,5-dihydroisoxazol-3-yl]benzyl}acetamide;
N-{2-fluoro-5-[5-(3,4,5-trichlorophenyl)-5-(trifluoromethyl)-4,5-dihydroisoxazol-3-yl]benzyl}-3-methylbutanamide;
N-{5-[5-(3,5-dichloro-4-fluorophenyl)-5-(trifluoromethyl)-4,5-dihydroisoxazol-3-yl]-2-fluorobenzyl}acetamide;
N-{2-chloro-5-[5-(3,4,5-trichlorophenyl)-5-(trifluoromethyl)-4,5-dihydroisoxazol-3-yl]benzyl}acetamide;
N-{2-chloro-5-[5-(3,4,5-trichlorophenyl)-5-(trifluoromethyl)-4,5-dihydroisoxazol-3-yl]benzyl}cyclopropanecarboxamide;
N-{2-chloro-5-[5-(3,4,5-trichlorophenyl)-5-(trifluoromethyl)-4,5-dihydroisoxazol-3-yl]benzyl}-2-methylpropanamide;
N-{2-chloro-5-[5-(3,5-dichloro-4-fluorophenyl)-5-(trifluoromethyl)-4,5-dihydroisoxazol-3-yl]benzyl}acetamide; and
N-(2-bromo-5-(5-(3,4,5-trichlorophenyl)-5-(trifluoromethyl)-4,5-dihydroisoxazol-3-yl)benzyl)acetamide, or a veterinarily acceptable salt thereof.

In still yet another aspect of the invention, Formula (1) compounds include:
N-{5-[5-(3,4,5-trichlorophenyl)-5-(trifluoromethyl)-4,5-dihydroisoxazol-3-yl]-2-fluorobenzyl}acetamide;
N-{2-fluoro-5-[5-(3,4,5-trichlorophenyl)-5-(trifluoromethyl)-4,5-dihydroisoxazol-3-yl]benzyl}-2-methylpropanamide;
N-{2-fluoro-5-[5-(3,4,5-trichlorophenyl)-5-(trifluoromethyl)-4,5-dihydroisoxazol-3-yl]benzyl}cyclopropanecarboxamide;
N-{2-fluoro-5-[5-(3,4,5-trichlorophenyl)-5-(trifluoromethyl)-4,5-dihydroisoxazol-3-yl]benzyl}cyclobutanecarboxamide;
N-{2-fluoro-5-[5-(3,4,5-trichlorophenyl)-5-(trifluoromethyl)-4,5-dihydroisoxazol-3-yl]benzyl}propanamide;
N-{5-[5-(3,5-dichloro-4-fluorophenyl)-5-(trifluoromethyl)-4,5-dihydroisoxazol-3-yl]-2-fluorobenzyl}acetamide;
N-{2-chloro-5-[5-(3,5-dichloro-4-fluorophenyl)-5-(trifluoromethyl)-4,5-dihydroisoxazol-3-yl]benzyl}acetamide; and
N-(2-bromo-5-(5-(3,4,5-trichlorophenyl)-5-(trifluoromethyl)-4,5-dihydroisoxazol-3-yl)benzyl)acetamide, or a veterinarily acceptable salt thereof.

Another embodiment of the present invention is a veterinary composition that comprises a) a Formula (1) compound, or a veterinarily acceptable salt thereof, and (b) a veterinarily acceptable excipient, diluent, or carrier. Preferrably, the composition comprises a therapeutically effective amount of a Formula (1) compound, or a veterinarily acceptable salt thereof, and a veterinarily acceptable excipient, diluent, or carrier.

The composition may comprise at least one additional veterinary agent. Preferred additional veterinary agents include endoparasiticides, endectocides, ectoparasiticides, insecticides, and anthelmintics.

In yet another aspect of the invention is the use of a Formula (1) compound for the manufacture of a medicament.

In yet another aspect of the invention is a method for treating or preventing a parasitic infection or infestation in an animal or bird that includes the step of administering to said animal or bird, in need of such treatment, a therapeutically effective amount of a compound of the present invention, or a veterinarily acceptable salt thereof. Formula (1) compounds, or a veterinarily acceptable salt thereof, or compositions thereof, may be administered orally, topically, and subcutaneously. More preferred, the compositions can be administered orally or topically.

In yet another aspect of the invention is a method for treating or preventing a parasitic infection or infestation in an animal or bird that includes the step of administering to said animal or bird, in need of such treatment, a therapeutically effective amount of a compound of the present invention, or a veterinarily acceptable salt thereof, in combination with at least one additional veterinary agent. Formula (1) compounds, or a veterinarily acceptable salt thereof, alone, or with an additional veterinary agent, or compositions thereof, may be administered orally, topically, and subcutaneously.

Specifically, animals include companion animals and livestock. More specifically, companion animals include cats, dogs, and horses. Even more specifically, companion animals include dogs and cats. Most specific companion animal is dog. Specific livestock include cattle, swine, sheep, goats, and bison; more specifically, livestock include cattle, swine, and sheep. Most specifically, livestock is cattle and sheep.

Specifically, birds are fowl. More specifically, fowl includes chicken, turkey, duck, and goose and most specific fowl is turkey and chicken.

Compounds of the present invention alone, or in combination with an additional veterinary agent may be administered as (a) a single veterinary composition which comprises a compound of the present invention, or a veterinarily acceptable salt thereof, and optionally, at least one additional veterinary agent as described herein and a veterinarily acceptable excipient, diluent, or carrier; or (b) two separate veterinary compositions comprising (i) a first composition comprising a compound of the present invention, or a veterinarily acceptable salt thereof, and a veterinarily acceptable excipient, diluent, or carrier, and (ii) a second composition comprising at least one additional veterinary agent, as described herein and a veterinarily acceptable excipient, diluent, or carrier. The veterinary compositions may be administered simultaneously or sequentially and in any order.

All of the recited WO patent publications and JP patent applications herein are incorporated by reference.

For the avoidance of doubt, it will be understood that throughout the application all references to veterinarily acceptable compounds and salts thereof, includes references topharmaceitcally acceptable compounds and salts thereof, or agriculturally acceptable compounds and salts, thereof. Furthermore it will be understood that throughout the application all references to veterinary activity includes references to pharmaceutical activity or agricultural activity.

### DEFINITIONS

For purposes of the present invention, as described and claimed herein, the following terms and phrases are defined as follows:

“Additional veterinary agent(s)” or “veterinary agent(s)” as used herein, unless otherwise indicated, refers to other veterinary compounds or products that provide a therapeutically effective amount of said agent(s) that are useful for the treatment of a parasitic infection or infestation in animals and birds, as described herein.

“Alkoxy”, as used herein, unless otherwise indicated, refers to an oxygen moiety having a further alkyl substituent. The alkyl portion (i.e., alkyl moiety) of an alkoxy group has the same definition as below. Non-limiting alkoxy examples include: —OCH₃, —OCH₂CH₃, and the like. The halo portion of an alkoxy group has the same definition as below. Non-limiting examples of halo alkoxy include: —OCH₂F, —OCHF₂, —OCF₃, —OCF₂Cl₃, and the like.

“Alkyl”, as used herein, unless otherwise indicated, includes saturated monovalent hydrocarbon alkane radicals of the general formula CₙH₂ₙ₊₁. The alkane radical may be straight or branched and may be unsubstituted or substituted. For example, the term “C₀-C₃ alkyl” or “C₁-C₈ alkyl” refers to a monovalent, straight or branched aliphatic group containing 0 to 3 or 1 to 8 carbon atoms, respectively. Non-exclusive examples of C₁-C₈ alkyl groups include, but are not limited to methyl, ethyl, propyl, isopropyl, sec-butyl, t-butyl, n-propyl, n-butyl, i-butyl, s-butyl, n-pentyl, 1-methylbutyl, 2-methylbutyl, 3-methylbutyl, neopentyl, 3,3-dimethylpropyl, 2-methylpentyl, 2,2-dimethylpentyl, hexyl, 3-ethylhexyl, heptyl, 4-ethylheptyl, octyl, and the like. Alkyl represented along with another term (e.g., alkylcycloalkyl (i.e., —CH₂cyclopentyl(methylcyclopentyl), —CH₂cyclobutyl, —(CH₂)₂cyclopropyl(ethylcyclopropyl), and the like. Said alkyl, cycloalkyl, and alkylcycloalkyl may be attached to the chemical moiety by any one of the carbon atoms of the aliphatic chain. The alkyl and alkylcycloalkyl moiety may be optionally substituted.

“Animal(s)”, as used herein, unless otherwise indicated, refers to an individual animal that is a member of the taxonomic class Mammalia. Non-exclusive examples of animals include companion animals and livestock.

“Compounds of the present invention”, as used herein, unless otherwise indicated, refers to Formula (1), (1A), (1B), (1C), and (1D) compounds, or a veterinarily acceptable salt thereof.

“Cycloalkyl”, as used herein, unless otherwise indicated, includes fully saturated or partially saturated carbocyclic alkyl moieties, wherein alkyl is as defined above. Non-limiting examples of partially saturated cycloalkyls include: cyclopropene, cyclobutene, cycloheptene, cyclooctene, cyclohepta-1,3-diene, and the like. Preferred cycloalkyls are 3- to 6-membered saturated monocyclic rings including cyclopropyl, cyclobutyl, cyclopentyl, and cyclohexyl. The cycloalkyl group may be attached to the chemical moiety by any one of the carbon atoms within the carbocyclic ring. Cycloalkyl groups are optionally substituted with at least one substituent.

“Fowl”, as used herein, unless otherwise indicated, refers to chicken, turkey, ducks, and geese, particularly chicken and turkey, and more particularly, chicken.

“Halogen” or “halo” as used herein, unless otherwise indicated, refers to either fluorine, chlorine, bromine or iodine. Further, when used in compound words such as “haloalkyl” or “haloalkoxy” said alkyl and alkoxy may be partially or fully substituted with halogen atoms which may be the same or different and said alkyl and alkoxy moiety has the same meaning as above and may be attached to the chemical moiety by any one of the carbon atoms of the aliphatic chain. Examples of “haloalkyl” include F₃C—, ClCH₂—, CF₃CH₂— and CF₃CCl₂—, and the like. The term “haloalkoxy” is defined analogously to the term “haloalkyl”. Examples of “haloalkoxy” include CF₃O—, CCl₃CH₂O—, HCF₂CH₂CH₂O— and CF₃CH₂O—, CF₂ClCH₂O—, and the like.

“Het” or “heteroaryl”, as used herein, unless otherwise indicated, refers to an aromatic monocyclic ring containing one or more heteroatoms each independently selected from N, S, or O, preferably from one to four nitrogen heteroatoms and optionally one oxygen or sulfur heteroatom. Non-exclusive examples of monocyclic rings include pyrolyl, pyrazolyl, oxazolyl, pyridinyl, triazolyl, tetrazolyl, pyridazinyl, pyrimidinyl, and the like. The Het group may be attached to the chemical moiety by any one of the carbon atoms or heteroatoms within the ring. The Het is optionally substituted.

“Insect(s)”, as used herein, unless otherwise indicated, refers to biting, chewing, or sucking insects. Non-exclusive examples of include biting flies (e.g., stable, horn, black, myasis, and horse), lice, midges, fleas, and the like.

“Parasite(s)”, as used herein, unless otherwise indicated, refers to endoparasites and ectoparasites. Endoparasites are parasites that live within the body of its host and include helminths (e.g., trematodes, cestodes, and nematodes) and protozoa. Ectoparasites are organisms of the Arthropoda phylum (arachnids and insects) which feed through or upon the skin of its host. Preferred arachnids are of the order Acarina, e.g., ticks and mites.

“Therapeutically effective amount”, as used herein, unless otherwise indicated, refers to an amount of the compounds of the present invention that (i) treat or prevent the particular parasitic infection or infestation, (ii) attenuates, ameliorates, or eliminates one or more symptoms of the particular parasitic infection or infestation, or (iii) prevents or delays the onset of one or more symptoms of the particular parasitic infection or infestation described herein.

“Treatment”, “treating”, and the like, as used herein, unless otherwise indicated, refers to reversing, alleviating, or inhibiting the parasitic infection, infestation, or condition. As used herein, these terms also encompass, depending on the condition of the animal, preventing or controlling the onset of a disorder or condition, or of symptoms associated with a disorder or condition, including reducing the severity of a disorder or condition or symptoms associated therewith prior to affliction with said infection or infestation. Thus, treatment can refer to administration of the compounds of the present invention to an animal that is not at the time of administration afflicted with the infection or infestation. Treating also encompasses preventing the recurrence of an infection or infestation or of symptoms associated therewith as well as references to “control” (e.g., kill, repel, expel, incapacitate, deter, eliminate, alleviate, minimize, and eradicate).

“Veterinarily or pharmaceutically acceptable” as used herein, unless otherwise indicated, indicates that the substance or composition must be compatible chemically and/or toxicologically, with the other ingredients comprising a formulation, composition, and/or the animal being treated therewith.

### DETAILED DESCRIPTION

The present invention provides Formula (1) compounds, or a veterinarily acceptable salt thereof, as well as veterinary compositions that are useful as antiparasitic agents for animals and birds, in particular, compounds that act as ectoparasiticides.

Compounds of the present invention may be synthesized by synthetic routes that include processes analogous to those well known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, “Reagents for Organic Synthesis”, 1; 19, Wiley, New York (1967, 1999 ed.), or Beilsteins Handbuch der organischen Chemie, 4, Aufl. ed. Springer-Verlag, Berlin, including supplements (also available via the Beilstein online database)).

Compounds of this invention can exist as one or more stereoisomers. The various stereoisomers include enantiomers, diastereomers and atropisomers. Included within the scope of the present invention are all stereoisomers such as enantiomers and diasteromers, all geometric isomers and tautomeric forms of the compounds of formula (I), including compounds exhibiting more than one type of isomerism, and mixtures of one or more thereof. The compounds of the invention may be present as a mixture of stereoisomers, individual stereoisomers or as an optically active form. For example, two possible enantiomers of Formula 1 are depicted as Formula 1a1 and Formula 1b1 involving the isoxazoline chiral center identified with an asterisk (*). One skilled in the art will appreciate that one stereoisomer may be more active and/or may exhibit beneficial effects when enriched relative to the other stereoisomer(s) or when separated from the other stereoisomer(s).

Conventional techniques for the preparation/isolation of individual enantiomers include chiral synthesis from a suitable optically pure precursor, stereoselective synthesis from a prochiral precursor or resolution of the racemate (or the racemate of a salt or derivative) using, for example, fractional crystallization or chiral high pressure liquid chromatography (HPLC). Reference is made herein to “Enantiomers, Racemates and Resolutions” J. Jacques and A. Collet, published by Wiley, NY, 1981; and “Handbook of Chiral Chemicals” chapter 8, Eds D. Ager and M. Dekker, ISBN:0-8247-1058-4. Geometric isomers may be separated by conventional techniques well known to those skilled in the art, for example, chromatography and fractional crystallisation.

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

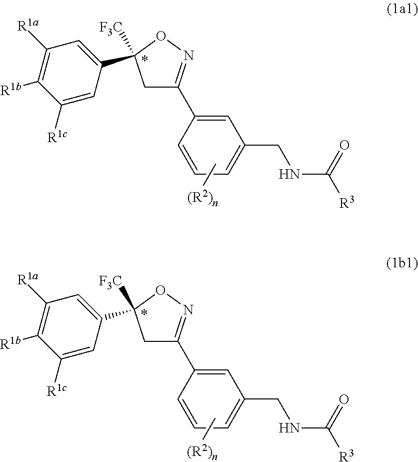

For illustrative purposes, the reaction schemes depicted below demonstrate potential routes for synthesizing key intermediates and compounds of the present invention. For a more detailed description of the individual reaction steps, see the Examples section below. Those skilled in the art will appreciate that other suitable starting materials, reagents, and synthetic routes may be used to synthesize the intermediates and compounds of the present invention and a variety of derivatives thereof. Further, many of the compounds prepared by the methods described below can be further modified in light of this disclosure using conventional chemistry. Schemes 1-7 outline the general procedures useful for the preparation of compounds of the present invention. It is to be understood, however, that the invention, as fully described herein and as recited in the claims, is not intended to be limited by the details of the following schemes or modes of preparation.

In the Schemes and Examples below, the following catalysts/reactants include: N,N-dimethyl formamide (DMF); N-bromo-succinimide (NBS); N-chloro-succinimide (NCS); acetonitrile (CAN), ethyl acetate (EtoAc), tetrahydrofuran (THF); triphenylphosphine (PPh₃); Dess-Martin periodinane (DMP); n-butyllithium (n-BuLi); dimethylsulfoxide (DMSO); triethylamine (TEA or NEt₃); ethyl acetate (EtOAc); bis(triphenylphosphine) palladium II chloride (Pd(PPh₃)₂Cl₂) from Strem; N,N,N′,N′-Tetramethyl-O-(7-azabenzotriazol-1-yl)uronium hexafluorophosphate (HATU) from Aldrich; bis(1,5-cyclooctadiene)di-mu-methoxyiiridum(I) (Ir[COD])₂) from Aldrich; 4,4,4′,4′,5,5,5′,5′-octamethyl[2,2′-bi-1,3,2-dioxaborolane] (B₂pin₂) from Aldrich; 4,4′-di-tert-butyl-2,2′-bypyridine (dtbpy) from Aldrich; N-Hydroxybenzotriazole (HOBT) from Aldrich, di-tert-butyl dicarbonate (BOC₂O) from Aldrich, 1-ethyl-3-(3-dimethyllaminopropyl)carbodiimide hydrochloride (EDC) from Aldrich, dimethyl acetamide (DMA), trifluoroacetic acid (TFA), and diphenylphosphoryl azide (DPPA).

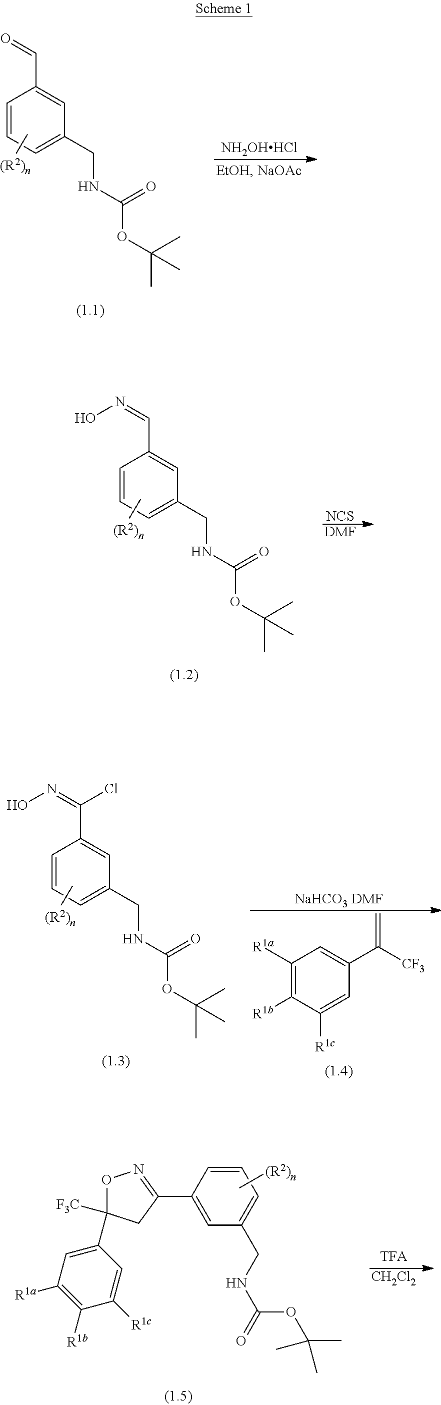

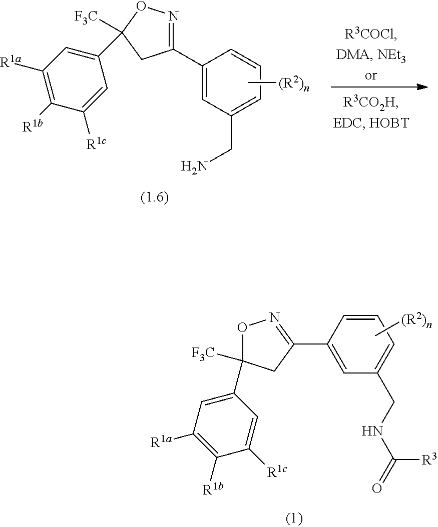

R^{1a}, R^{1b}, R^{1c}, R², and n are as defined herein.

In Scheme 1, intermediate (1.2) compounds can be prepared by reacting intermediate (1.1) compounds with N-hydroxylamine in the presence of a base such as sodium acetate in a solvent such as ethanol. Chlorination of intermediate (1.2) compounds can be accomplished with N-chlorosuccinimide (NCS) in a solvent such as DMF at temperatures between about 0° C. and 50° C. to provide intermediate (1.3) compounds. The reaction of intermediate (1.3) compounds with intermediate (1.4) compounds in the presence of a base such as sodium hydrogen carbonate and in a solvent such as ethyl acetate, THF or DMF can give intermediate (1.5) compounds. Deprotection of the intermediate (1.5) compound can be carried out using standard conditions, for example with TFA in methylene chloride to give intermediate (1.6) compounds. Compounds of Formula (1) can be prepared by reacting the intermediate (1.6) compounds with an acyl chloride in the presence of a base such as triethylamine or pyridine in a solvent such as methylene chloride or DMF. Formula (1) compounds can also be prepared by reacting intermediate (1.6) compounds with a carboxylic acid in the presence of a suitiable peptide coupling reagent such as EDC, dicyclohexylcarbodiimide (DCC), HBTU, HATU, or N,N′-diisopropylcarbodiimide (DIC) to afford the Formula (1) compounds. In addition, Formula (1) compounds can also be prepared by reaction of intermediate (1.6) compounds with anhydrides of carboxylic acids in an aprotic solvent such as THF, methylene chloride or DMF.

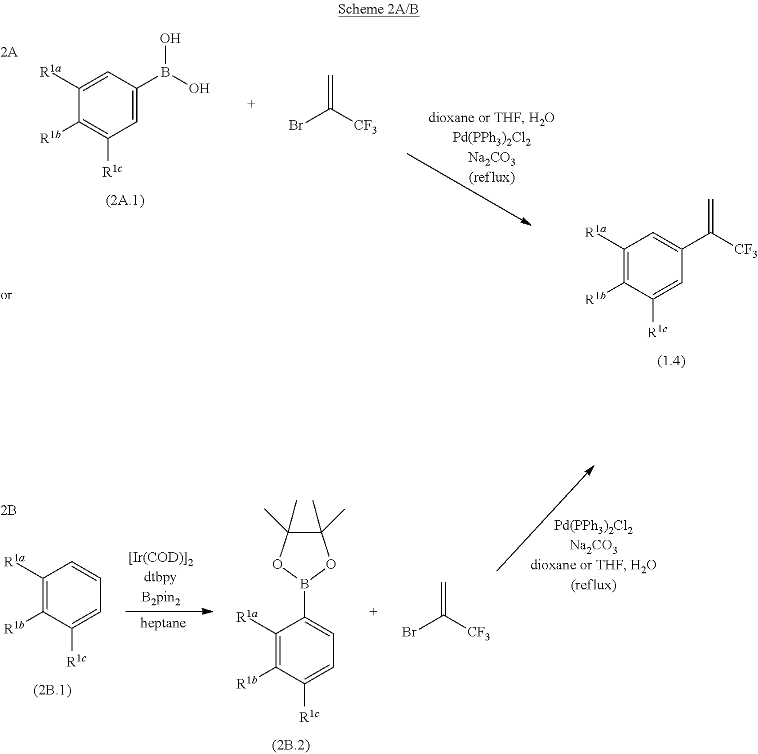

R^{1a}, R^{1b}, and R^{1c} are as defined herein.

Scheme 2 describes the synthesis of intermediate compounds 1.4. The requisite organoborates can be prepared as boronate ester intermediates (2B.2) from literature methods (Org. Lett. 2007, 9, 761-764) or purchased as boronic acids (2A.1) such as 3,5-dichloroboronic acid from Aldrich. Intermediate 2A.1 or 2B.2 compounds can be added to dioxane or THF and water, followed by 2-bromo-3,3,3-trifluoropropene, potassium carbonate, and bis(triphenylphosphine) palladium II chloride to afford intermediate (1.4) compounds.

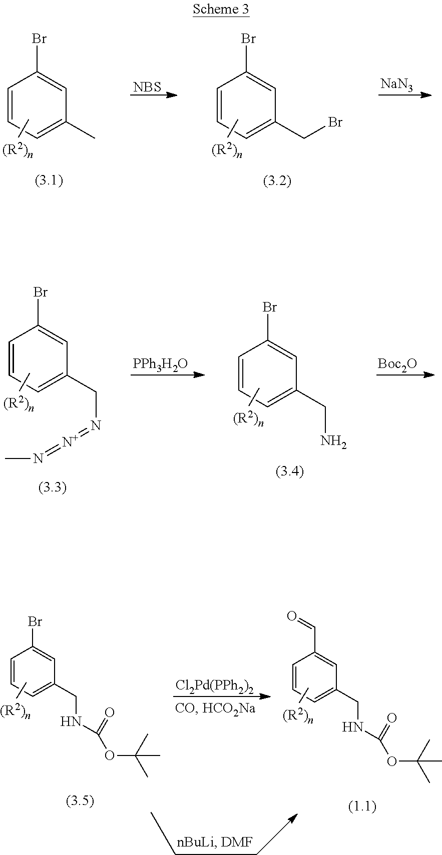

R² and n are as defined herein.

Formula (1.1) compounds can be obtained through a process shown in Scheme 3. Intermediate (3.1) compounds are available from commercial sources.

Treatment of intermediate (3.1) compounds with NBS and a catalytic amount of benzoyl peroxide in a solvent such as CCl₄ will yield compounds of intermediate (3.2). Treatment of intermediate (3.2) compounds with sodium azide in a solvent such as DMSO will yield compounds of intermediate (3.3). Intermediate (3.4) compounds can be prepared by treating compounds of intermediate (3.3) with triphenyl phosphine and water in a solvent such as THF. Alternatively, compounds of intermediate (3.4) can be obtained after reduction of intermediate (3.3) compounds with hydrogen in the presence of a catalyst such as palladium on carbon in a suitable solvent such as ethanol. Intermediate (3.5) compounds can be obtained by reacting intermediate (3.4) compounds with Boc-anhydride in the presence of one or more equavalents of base such as triethylamine in a suitable solvent such as methylene chloride. Formula (1.1) compounds can be obtained by reacting the intermediate (3.5) compounds with a catalyst such as palladium dichlorobistriphenylphosphine in the presence of carbon monoxide and sodium formate in a solvent system such as DMF at elevated temperature of 80° C. to 100° C., as described in US patent application US2004/0138271. Intermediate (3.5) compounds can also be obtained after treating intermediate (3.4) compounds with two or more equivalents of an alkyl lithium followed by quench with DMF. The reaction is carried out at a low temperature (−78° C.) in a solvent such as THF.

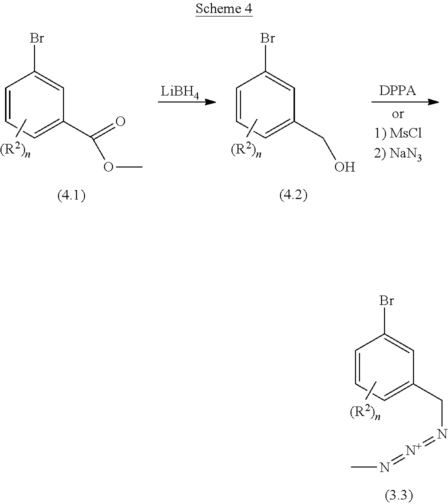

R² and n are as defined herein.

Intermediate compounds of formula (3.3) may also be prepared as shown in Scheme 4. Commercially available benzoate esters can be reacted with a hydride reducing agent such as lithium borohydride to give compounds of formula (4.2). Compounds of formula (3-3) may be prepared by reacting compounds of formula (4-2) with diphenyl phosphoryl azide or through the conversion of the hydroxyl to a leaving group (e.g., methane sulfonate, Cl, or Br) and displacement with sodium azide.

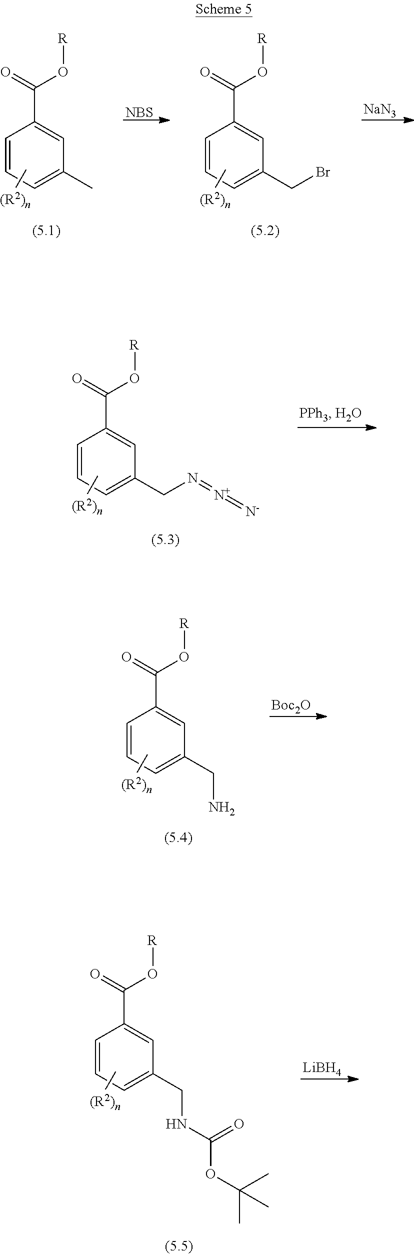

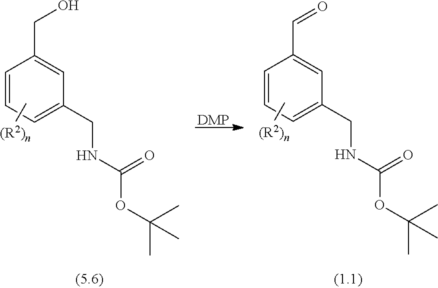

R² and n are as defined herein.

Compounds having formula (1.1) may also be prepared from commercially available compounds of (5.1) as shown in Scheme 5. The compound of formula (5-2) may be prepared by reacting (5.1) with N-bromosuccinimide (NBS) in the presence of a catalytic amount of benzoyl peroxide in a organic solvent such as chloroform or carbon tetrachloride. Compounds of formula (5.3) may be had after treatment of (5.2) with one equivalent of sodium azide in a solvent such as DMSO at a temperature not to exceed 50° C. Compounds of formula (5.4) maybe obtained after treatment of (5.3) with triphenylphosphine and water in a solvent such as THF. Alternatively compounds of formula (5.4) may be prepared by reduction of compounds of formula (5.3) with hydrogen in the presence of a catalyst such as palladium on carbon in a solvent such as ethanol. Compounds of formula (5.5) may be prepared by treatment of (5.4) with di-tert-butyldicarbonate in the presence of a base such as triethylamine in a solvent such as methylene chloride. Compounds of formula (5.6) may be prepared by reaction of (5.5) with a hydride reducing agent such as lithium borohydride in a dual solvent system of THF and methanol. Compounds of formula (1.1) may be prepared by oxidation of (5.6) with Dess-Martin periodinane (1,1,1-Triacetoxy-1,1-dihydro-1,2-benziodoxol-3(1H)-one).

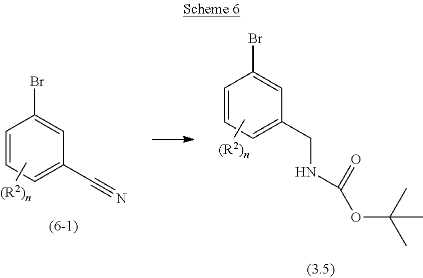

R² and n are as defined herein.

Compounds of formula (3-5) can also be prepared from the corresponding commercially available nitriles, as described in Tetrahedron 59, 5417, (2003) and Biorganic and Medicinal Chemistry Letters, 18, 2362, (2008) via a one-pot reduction-protection strategy.

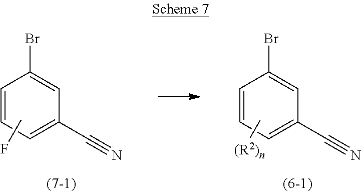

R² and n are as defined herein.

Compounds of formula 6-1 can be prepared via displacement of an atom such as fluorine as shown in Scheme 7 (Tetrahedron Letters, 50(12), 1286-1289, (2009)).

One, skilled in the art will recognize that, in some cases, after the introduction of a given reagent as it is depicted in the schemes, it may be necessary to perform additional routine synthetic steps not described in detail to complete the synthesis of Formula (1) compounds.

One skilled in the art will also recognize that Formula (1) compounds and the intermediates described herein can be subjected to various electrophilic, nucleophilic, radical, organometallic, oxidation, and reduction reactions to add substituents or modify existing substituents.

Veterinarily acceptable salts of Formula (1), (1A), (1B), (1C), or (1 D) compounds include the acid addition and base salts thereof. Suitable acid addition salts are formed from acids, which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, laurate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

The veterinarily acceptable acid addition salts of certain of the Formula (1), (1A), (1B), (1C), (1D), compounds may also be prepared in a conventional manner. For example, a solution of a free base may be treated with the appropriate acid, either neat or in a suitable solvent, and the resulting salt isolated either by filtration or by evaporation under reduced pressure of the reaction solvent. For a review on suitable salts, see “Handbook of Pharmaceutical Salts: Properties, Selection, and Use” by Stahl and Wermuth (Wiley-VCH, Weinheim, Germany, 2002).

The compounds of the invention may exist in both unsolvated and solvated forms. The term ‘solvate’ is used herein to describe a molecular complex comprising the compound of the invention and one or more veterinarily acceptable solvent molecules, for example, ethanol. The term ‘hydrate’ is employed when said solvent is water. Veterinarily acceptable solvates in accordance with the invention include those wherein the solvent of crystallization may be isotopically substituted, e.g. D₂O, d₆-acetone, d₆-DMSO.

Hereinafter and throughout the application all references to Formula (1), (1A), (1B), (1C), (1D), compounds include references to salts, solvates and complexes thereof and to solvates and complexes of salts thereof.

As stated, the invention includes all polymorphs of the Formula (1), (1A), (1B), (1C), (1D) compounds as herein defined.

The present invention includes all veterinarily acceptable isotopically-labelled Formula (1) compounds wherein one or more atoms are replaced by atoms having the same atomic number, but an atomic mass or mass number different from the atomic mass or mass number usually found in nature.

Examples of isotopes suitable for inclusion in the compounds of the present invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁶N, oxygen, such as ¹⁵O, ¹⁷O and ¹⁸O, and sulphur, such as ³⁵S.

The skilled person will appreciate that the compounds of the present invention could be made by methods other than those herein described as incorporated herein by reference, by adaptation of the methods herein described and/or adaptation of methods known in the art, for example the art described herein, or using standard textbooks such as “Comprehensive Organic Transformations—A Guide to Functional Group Transformations”, RC Larock, Wiley-VCH (1999 or later editions).

The Formula (1) compounds are useful as ectoparasitic agents, therefore, another embodiment of the present invention is a veterinary composition comprising a therapeutically effective amount of a Formula (1) compound, or a veterinarily acceptable salt thereof, and a veterinarily acceptable excipient, diluent or carrier. The compounds of the present invention (including the compositions and processes used therein) may also be used in the manufacture of a medicament for the therapeutic applications described herein.

A typical formulation is prepared by mixing a Formula (1) compound with a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. Solvents are generally selected based on solvents recognized by persons skilled in the art as safe to be administered to a animal. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or veterinary composition thereof) or aid in the manufacturing of the veterinary product (i.e., medicament).

The formulations can be prepared using conventional dissolution and mixing procedures. Such compositions and methods for their preparation may be found, for example, in ‘Remington's Veterinary Sciences’, 19th Edition (Mack Publishing Company, 1995; and “Veterinary Dosage Forms: Tablets, Vol. 1”, by H. Lieberman and L. Lachman, Marcel Dekker, N.Y., 1980 (ISBN 0-8247-6918-X). For example, the bulk drug substance (i.e., compound of the present invention or stabilized form of the compound (e.g., complex with a cyclodextrin derivative or other known complexation agent)) is dissolved in a suitable solvent in the presence of one or more other excipients. The compounds of the present invention are typically formulated into veterinary dosage forms to provide an easily controllable dosage form for administration.

The compounds may be administered alone or in a formulation appropriate to the specific use envisaged, the particular species of host animal or bird being treated and the parasite involved. Generally, they will be administered as a formulation in association with one or more veterinarily acceptable excipients, diluents, or carriers. The term “excipient”, “diluent” or “carrier” is used herein to describe any ingredient other than the Formula (1) compounds or any additional antiparasitic agent. The choice of excipient, diluent, or carrier will to a large extent depend on factors such as the particular mode of administration, the effect of the excipient, carrier, or diluent on solubility and stability, and the nature of the dosage form.

The methods by which the compounds of the present invention may be administered include oral, topical, and subcutaneous administration. The invention contemplates monthly administration of the described compositions.

The Formula (1) compounds can be administered orally by capsule, bolus, tablet, powders, lozenges, chews, multi and nanoparticulates, gels, solid solution, films, sprays, or liquid form. This is a preferred method of administration and as such it is desirable to develop active Formula (1) compounds that are particularly suited to such formulations. Such formulations may be employed as fillers in soft or hard capsules and typically comprise a carrier, for example, water, ethanol, polyethylene glycol, N-methylpyrrolidone, propylene glycol, methylcellulose, or a suitable oil, and one or more emulsifying agents and/or suspending agents. Liquid forms include suspensions, solutions, syrups, drenches and elixirs. Liquid formulations may also be prepared by the reconstitution of a solid, for example, from a sachet. Oral drenches are commonly prepared by dissolving or suspending the active ingredient in a suitable medium. This is a preferred method of administration and as such it is desirable to develop active Formula (1) compounds that are particularly suited to such formulations. Oral formulations can comprise from about 0.5 mg/kg to 50 mg/kg of a Formula (1) compound, and preferably about 1 mg/kg to 30 mg/kg of a Formula (1) compound.

The compounds may be administered topically to the skin or mucosa, that is dermally or transdermally. This is a preferred method of administration and as such it is desirable to develop active Formula (1) compounds that are particularly suited to such formulations, for example liquid forms. Typical formulations for this purpose include pour-on, spot-on, multi-spot-on, stripe-on, comb-on, roll-on, dip, spray, mousse, shampoo, powder formulation, gels, hydrogels, lotions, solutions, creams, ointments, dusting powders, dressings, foams, films, skin patches, wafers, implants, sponges, fibers, bandages and micro emulsions. Liposomes may also be used. Typical carriers include alcohol, water, mineral oil, liquid petrolatum, white petrolatum, glycerin, N-methyl formamide, glycol monomethyl ethers, polyethylene glycol, propylene glycol, and the like. Penetration enhancers may be incorporated—see, for example, J Pharm Sci, 88 (10), 955-958 by Finnin and Morgan (October 1999). Pour-on or spot-on formulations may be prepared by dissolving the active ingredients in an acceptable liquid carrier vehicle such as butyl digol, liquid paraffin or a non-volatile ester, optionally with the addition of a volatile component such as propan-2-ol or a glycol ether. Alternatively, pour-on, spot-on or spray formulations can be prepared by encapsulation, to leave a residue of active agent on the surface of the animal, this effect may ensure that the Formula (1) compounds have increased persistence of action and are more durable, for example they may be more water fast. Topical formulations of the combination contemplated herein can comprise from about 0.5 mg/kg to 50 mg/kg of a Formula (1) compound, and preferably about 1 mg/kg to 10 mg/kg of a Formula (1) compound.

The compounds of the present invention can also be administered topically via a support matrix for example, a synthetic or natural resin, plastic, cloth, leather, or other such polymeric system in the shape of a collar or ear tag. Said collar or ear tag may be coated, impregnated, layered, by any means so as to provide a veterinarily acceptable amount of a compound of the present invention alone, or with a veterinarily acceptable excipient, diluent, or carrier, and optionally an additional veterinary agent, or veterinarily acceptable salt thereof.

The compositions suitable for spot-on application according to the invention can be prepared by conventional mixing means. The volume of the applied composition can be from about 0.5 mL/kg to 5 mL/kg and preferably from about 1 mL/kg to 3 mL/kg.

Agents may be added to the formulations of the present invention to improve the persistence of such formulations on the surface of the animal to which they are applied, for example to improve their persistence on the coat of the animal. It is particularly preferred to include such agents in a formulation which is to be applied as a pour-on or spot-on formulation. Examples of such agents include acrylic copolymers and in particular fluorinated acrylic copolymers. A particular suitable reagent is the trademark reagent “Foraperle” (Redline Products Inc, Texas, USA).

Certain topical formulations may include unpalatable additives to minimize oral exposure.

Subcutaneous injectable formulations may be prepared in the form of a sterile solution, which may contain other substances, for example enough salts or glucose to make the solution isotonic with blood. Acceptable liquid carriers include vegetable oils such as sesame oil, glycerides such as triacetin, esters such as benzyl benzoate, isopropyl myristate and fatty acid derivatives of propylene glycol, as well as organic solvents such as pyrrolidin-2-one and glycerol formal. The formulations are prepared by dissolving or suspending compounds of the instant invention alone or with an additional veterinary agent in the liquid carrier such that the final formulation contains from about 0.01 to 10% by weight of the active ingredients.

Suitable devices for subcutaneous administration include needle (including micro needle) injectors, needle-free injectors and infusion techniques. Subcutaneous formulations are typically aqueous solutions which may contain excipients such as salts, carbohydrates and buffering agents (preferably to a pH of from 3 to 9), but, for some applications, they may be more suitably formulated as a sterile non-aqueous solution or as a dry powder form to be used in conjunction with a suitable vehicle such as sterile, pyrogen-free water. The preparation of subcutaneous formulations under sterile conditions, for example, by lyophilisation, may readily be accomplished using standard veterinary techniques well known to those skilled in the art. The solubility of compounds of Formula (1) used in the preparation of subcutaneous solutions may be increased by the use of appropriate formulation techniques, such as the incorporation of solubility-enhancing agents.

Such formulations are prepared in a conventional manner in accordance with standard medicinal or veterinary practice. Further, these formulations will vary with regard to the weight of active compound contained therein, depending on the species of host animal to be treated, the severity and type of infection or infestation, and the body weight of the animal.

As described herein, compounds of the present invention may be administered alone or in combination with at least one additional veterinary agent including insecticides, acaricides, anthelmintics, fungicides, nematocides, antiprotozoals, bactericides, and growth regulators to form a multi-component agent giving an even broader spectrum of veterinary utility. Thus, the present invention also pertains to a composition comprising an effective amount of a Formula (1) compound, or a veterinarily acceptable salt thereof, and an effective amount of at least one additional veterinary agent and can further comprise one or more of a veterinarily acceptable excipient, diluent, or carrier.

The following list of additional veterinary agents together with which the compounds of the present invention can be used is intended to illustrate the possible combinations, but not to impose any limitation. Non-limiting examples of additional veterinary agents include: amitraz, arylpyrazoles as recited in publications WO1998/24767 and WO2005/060749, amino acetonitriles, anthelmintics (e.g., albendazole, cambendazole, fenbendazole, flubendazole, mebendazole, octadepsipeptides, oxfendazole, oxibendazole, paraherquamide, parbendazole, piperazines, praziquantel, thiabendazole, tetramisole, triclabendazole, levamisole, pyrantel pamoate, oxantel, morantel, and the like), avermectins (e.g., abamectin, doramectin, emamectin, eprinomectin, ivermectin, moxidectin, selamectin, and the like), DEET, demiditraz, diethylcarbamazine, fipronil, insect growth regulators (e.g., hydroprene, kinoprene, methoprene, and the like), metaflumizone, milbemycin and derivatives thereof (e.g., milbemycin oxime), niclosamide, permethrin, pyrethrins, pyriproxyfen, spinosad, and the like. In certain instances, combinations of a Formula (1) compound with an additional veterinary agent(s) can result in a greater-than-additive effect. Reducing the quantity of active ingredients released in the environment while ensuring effective pest control is always desirable.

It may be desirable to administer a compound of the present invention, or a veterinarily acceptable salt thereof, alone or in a composition comprising a veterinarily acceptable excipient, diluent, or carrier, for example, for the purpose of treating a particular parasitic infection or infestation or condition associated therewith. It is within the scope of the present invention that two or more veterinary compositions, at least one of which contains a Formula (1) compound in accordance with the invention, and the other, an additional veterinary agent, may conveniently be combined in the form of a kit suitable for coadministration of the compositions.

The compounds of the present invention (including the compositions and processes used therein) may also be used in the manufacture of a medicament for the therapeutic applications described herein.

The compounds of the present invention, or a veterinarily acceptable salt thereof, and compositions comprising a therapeutically effective amount of a Formula (1) compound and a veterinarily acceptable excipient, diluent, or carrier are useful as ectoparasiticides for the control and treatment of infections or infestations manifested by said ectoparasite in an animal or bird. The compounds of the present invention have utility as an ectoparasiticide, in particular, as an acaricide and insecticide. They may, in particular, be used in the fields of veterinary medicine, livestock husbandry and the maintenance of public health: against acarids and insects which are parasitic upon vertebrates, particularly warm-blooded vertebrates, including companion animals, livestock, and birds. Some non-limiting examples of acaride and insect parasites include: ticks (e.g., Ixodes spp., Rhipicephalus spp., Boophilus spp., Amblyomma spp., Hyalomma spp., Haemaphysalis spp., Dermacentor spp., Ornithodorus spp., and the like); mites (e.g., Dermanyssus spp., Sarcoptes spp., Psoroptes spp., Chorioptes spp., Demodex spp., and the like); chewing and sucking lice (e.g., Damalinia spp., Linognathus spp., and the like); fleas (e.g., Siphonaptera spp., Ctenocephalides spp., and the like); and biting flies and midges (e.g., Tabanidae spp., Haematobia spp., Stomoxys spp., Dermatobia spp., Simuliidae spp., Ceratopogonidae spp., Psychodidae spp., and the like).

The compounds of the present invention and compositions comprising compounds of the present invention in conjunction with at least one other veterinary agent are of particular value in the control of ectoparasites, endoparasites, and insects which are injurious to, or spread or act as vectors of diseases in companion animals, livestock, and birds. The ectoparasites, insects, and endoparasites which can be treated with a combination of a Formula (1) compound and an additional veterinary agent include those as herein before described and including helminthes of the phylum platyhelminthes (e.g., trematodes, eucestoda, and cestoda), and nemathelminthes (e.g., nematodes).

Any of the compounds of the present invention, or a suitable combination of a compound of the present invention and optionally, with at least one additional veterinary agent may be administered directly to the animal or bird and/or indirectly by applying it to the local environment in which the animal or bird dwells (such as bedding, enclosures, and the like). Direct administration includes contacting the skin, fur, or feathers of a subject animal or bird with the compound(s), or by feeding or injecting the compounds into the animal or bird.

The Formula (1) compounds, or a veterinarily acceptable salt thereof, and combinations with at least one additional veterinary agent, as described herein, are of value for the treatment and control of the various lifecycle stages of insects and parasites including egg, nymph, larvae, juvenile and adult stages.

The present invention also relates to a method of administering a compound of the present invention alone or in combination with at least one additional veterinary agent, and optionally a veterinarily acceptable excipient, diluent, or carrier, to animals or birds in good health comprising the application to said animal or bird to reduce or eliminate the potential for human parasitic infection or infestation from parasities carried by the animal or bird and to improve the environment in which the animals, birds, and humans inhabit.

The reactions set forth below were done generally under a positive pressure of argon or nitrogen or with a drying tube, at ambient temperature (unless otherwise stated), in anhydrous solvents, and the reaction flasks were fitted with rubber septa for the introduction of substrates and reagents via syringe. Glassware was oven dried and/or heat dried. Analytical thin layer chromatography (TLC) was performed using glass-backed silica gel 60 F 254 precoated plates and eluted with appropriate solvent ratios (v/v). Reactions were assayed by TLC or LCMS and terminated as judged by the consumption of starting material. Visualization of the TLC plates was done with UV light (254 nM wavelength) or with an appropriate TLC visualizing solvent and activated with heat. Flash column chromatography (Still et al., J. Org. Chem. 43, 2923, (1978) was performed using silica gel (RediSep Rf) or various MPLC systems, such as Biotage or ISCO purification system.

Conventional methods and/or techniques of separation and purification known to one of ordinary skill in the art can be used to isolate the compounds of the present invention, as well as the various intermediates related thereto. Such techniques will be well-known to one of ordinary skill in the art and may include, for example, all types of chromatography (high pressure liquid chromatography (HPLC), column chromatography using common adsorbents such as silica gel, and thin-layer chromatography (TLC), recrystallization, and differential (i.e., liquid-liquid) extraction techniques.

The compound structures in the examples below were confirmed by one or more of the following methods: proton magnetic resonance spectroscopy, and mass spectroscopy. Proton magnetic resonance (¹H NMR) spectra were determined using a Bruker spectrometer operating at a field strength of 400 megahertz (MHz). Chemical shifts are reported in parts per million (PPM, δ) downfield from an internal tetramethylsilane standard. Mass spectra (MS) data were obtained using Agilent mass spectrometer with atmospheric pressure chemical ionization. Method: Acquity HPLC with chromatography performed on a Waters BEH C18 column (2.1×50 mm, 1.7 μm) at 50° C. The mobile phase was a binary gradient of acetonitrile (containing 0.1% trifluoroacetic acid) and water (5-100%).

Embodiments of the present invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

The following examples provide a more detailed description of the process conditions. It is to be understood, however, that the invention, as fully described herein and as recited in the claims, is not intended to be limited by the details of the following schemes or modes of preparation.

### Preparation 1. 1,2,3-trichloro-5-(1,1,1-trifluoroprop-2-en-2-yl)benzene

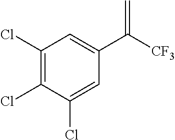

To a mixture of 25.0 g (131 mmol) of 3,4,5-trichlorobenzeneboronic acid (2A.1) in 200 mL of THF and 100 mL of water, 2-bromo-3,3,3-trifluoropropene, potassium carbonate, and bis(triphenylphosphine) palladium II chloride were added, and stirred under reflux overnight. The reaction mixture was partitioned between water and ethyl acetate, the organics were washed with brine and dried over MgSO₄, filtered, and the concentrate yielded an orange solid (7 g). The crude material was absorbed onto silica gel and purified by column chromatography, 0-10% acetone/heptane, 120 g silica. The relevant fractions were combined and concentrated to afford the title compound as a colorless oil (5.35 g). ¹H NMR (CDCl₃) δ 5.85 (1H), 6.07 (1H), 7.48 (2H).

### Preparation 2. 1,3-dichloro-2-fluoro-5-(1,1,1-trifluoroprop-2-en-2-yl)benzene

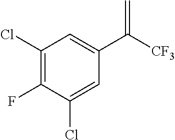

To a stirred solution of 2-bromo-3,3,3-trifluoropropene (2.65 g, 15.1 mmol) and 2-(3,5-dichloro-4-fluorophenyl)-4,4,5,5-tetramethyl-1,3,2-dioxaborolane (2A.1) (4.4 g, 15.1 mmol) in 1,4-dioxane (60 mL) was added Na₂CO₃ (4.02 g, 38 mmol) and water (20 mL). Next, bis(triphenylphosphine) palladium II chloride (220 mg, 0.3 mmol) was added and the reaction mixture was heated to 80° C. for 18 hours. The reaction mixture was cooled, filtered, and concentrated under reduced pressure to remove dioxane. The residue was diluted with water (100 mL) and extracted with EtOAc (2×125 mL), dried (Na₂SO₄), and concentrated under vacuum. Crude material was purified on silica gel with 100% heptane to afford the intermediate as a clear oil (1.8 g, 47%). ¹H NMR (CDCl₃): δ 7.43 (2H), 6.07 (1H), 5.82 (1H).

### Preparation 3. tert-butyl (5-bromo-2-fluorobenzyl)carbamate

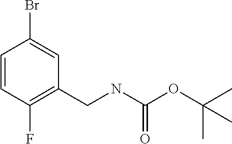

To a cold (0° C.) solution of 1-(5-bromo-2-fluorophenyl)methanamine hydrochloride (10 g, 41.6 mmol) in methylene chloride (100 mL) was added di-tert-butyl dicarbonate (9.07 g, 41.6 mmol) followed by the addition of triethylamine (8.4 g, 83.2 mmol). The solution was stirred at 0° C. for 30 minutes and then for two hours at room temperature. The reaction was washed with water (2×25 mL) and concentrated using rotary evaporation to give crude product as a viscuous oil. The product was purified on silica gel using a gradient of ethyl acetate in hexanes to give the title compound as a viscuous colorless oil. (12.51 g, 41.6 mmol, 99%) (¹H NMR (CDCl₃) δ ppm: 7.46 (1H), 7.40 (1H), 6.92 (1H), 4.90 (1H), 4.30 (1H), 1.48 (9H).

### Preparation 4. tert-butyl (2-fluoro-5-formylbenzyl)carbamate

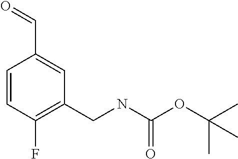

An amount of (5-bromo-2-fluorobenzyl)carbamate (10.2 g, 33.5 mmol) was dissolved in anhydrous THF (80 mL) and the solution was cooled to −78° C. in a dry ice acetone bath. While keeping the reaction under an inert atmosphere of nitrogen, n-BuLi (44 mL, 1.6M in hexanes, 70.3 mmol) was added dropwise, via addition funnel, over a 30 minute period while maintaining the temperature at −78° C. The solution was stirred for 10 minutes more before DMF (4.9 g, 68 mmol) was added all at once. The cold bath was removed and the reaction was allowed to equilibrate to room temperature over two hours. The reaction was cooled to 0° C. and quenched by the addition of saturated aqueous ammonium chloride (50 mL). The layers were stirred together for 30 minutes and then allowed to separate. The organic phase was collected, dried over sodium sulfate and concentrated using rotary evaporation at low pressure to provide a viscous oil. The oil was subjected to flash column chromatography using an ethyl acetate gradient in hexanes to afford the title compound as a viscous oil. (6.78 g, 80%) ¹H NMR (400 MHz, CDCl₃) δ ppm 1.48 (s, 9H) 4.45 (d, J=5.56 Hz, 2H) 4.99-5.06 (br, 1H) 7.17-7.25 (m, 1H) 7.82-7.86 (m, 1H) 7.92 (d, J=5.05 Hz, 1H) 9.97 (s, 1H).

### Preparation 5. tert-butyl{2-fluoro-5-[(E/Z)-(hydroxyimino)methyl]-benzyl}-carbamate

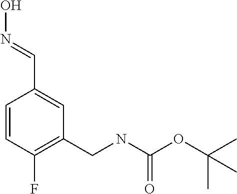

To an ethanolic (20 mL) mixture of tert-butyl (2-fluoro-5-formylbenzyl)carbamate (1.0 g, 3.9 mmol) was added hydroxylamine hydrochloride (1.37 g, 19.7 mmol) and sodium acetate (1.62 g, 19.7 mmol). The mixture was stirred at room temperature for four hours. The volatiles were removed by rotary evaporation at low pressure. Water (40 mL) was added to the flask to suspend the product. After stirring the mixture for 30 minutes, the white solid was collected by suction filtration, washed with water (2×20 mL) and air dried to afford the intermediate which was used in the next step without additional purification. ¹H NMR (400 MHz, CDCl₃) δ ppm 1.48 (s, 9H) 4.40 (d, J=5.05 Hz, 2 H) 4.93-5.02 (br s, 1H) 7.04-7.09 (m, 1H) 7.49 (br m, 1H) 7.57 (dd, J=7.20, 2.15 Hz, 1H) 8.10 (s, 1H).

### Preparation 6. tert-butyl {545-(3,4,5-trichlorophenyl)-5-(trifluoromethyl)-4,5-dihydroisoxazol-3-yl]-2-fluorobenzyl}carbamate

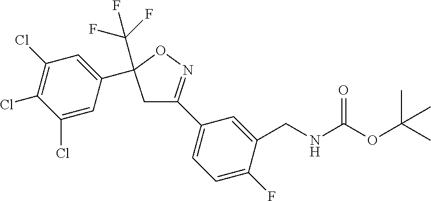

To a DMF (5 mL) solution of tert-butyl {2-fluoro-5-[(E/Z)-(hydroxyimino)methyl]benzyl}carbamate (Preparation 5, 250 mg, 0.826 mmols) was added n-chlorosuccinimide (115 mg, 0.858) in three equal portions over 30 minutes with approximately ten minutes between each addition. The reaction mixture was stirred in an atmosphere of nitrogen for twelve hours. To the crude reaction mixture was added 1,2,3-trichloro-5-(1,1,1-trifluoroprop-2-en-2-yl)benzene (Preparation 1, 228 mg, 0.826 mmol) and solid sodium hydrogen carbonate (300 mg). The mixture was stirred at room temperature for 24 hours. The reaction mixture was partitioned between water (10 mL) and EtOAc (40 mL). The organic phase was washed successively with water (3×15 mL) dried (sodium sulfate), and the solvent distilled off at low pressure to give the crude product as a viscous colorless oil. The product was purified on silica gel (EtOAc gradient in hexanes) to afford the title compound as an amorphous glass (221 mg, 49%). m/z (Cl) 443 [M+H]⁺ (the Boc group is lost upon ionization).

### Preparation 7. 1-{2-fluoro-5-[5-(3,4,5-trichlorophenyl)-5-(trifluoromethyl)-4,5-dihydroisoxazol-3-yl]phenyl}methanamine hydrochloride

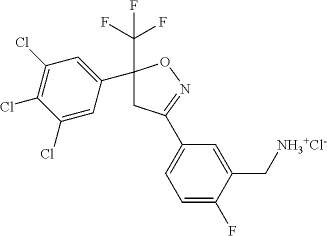

To a methylene chloride (10 mL) solution of tert-butyl {5-[5-(3,4,5-trichlorophenyl)-5-(trifluoromethyl)-4,5-dihydroisoxazol-3-yl]-2-fluorobenzyl}-carbamate (Preparation 6) was added TFA (3 mL). The reaction was stirred over-night at room temperature. The volatiles were distilled off at low pressure. Excess TFA was removed by performing several evaporation cycles using acetonitrile (3×20 mL). The crude residue was dissolved in EtOAc (40 mL) and while the mixture was stirred 4N HCl in dioxane (5 mL) was added. A white precipiate formed (HCl salt). The mixture was stirred in a closed vessel for ninety minutes. The white solid was captured using suction filtration to afford the title compound. m/z (Cl) 443 [M+H]⁺.

### Preparation 8: ethyl 3-(2-fluoro-5-(5-(3,4,5-trichlorophenyl)-5-(trifluoromethyl)-4,5-dihydroisoxazol-3-yl)benzylamino)-3-oxopropanoate

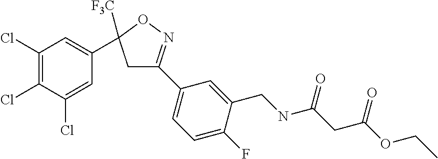

1-{2-fluoro-5-[5-(3,4,5-trichlorophenyl)-5-(trifluoromethyl)-4,5-dihydroisoxazol-3-yl]phenyl}methanamine hydrochloride (6 g, 13.6 mmol) was dissolved in dichloromethane (60 mL), placed under an inert atmosphere (N₂) and cooled to 0° C. The reaction mixture was treated with triethylamine (0.69 mL, 4.9 mmol), and ethyl malonyl chloride (0.63 mL, 4.9 mmol) dropwise. The reaction was allowed to warm to ambient temperature, and stirred for 1 hour. The reaction mixture was purified by column chromatography, silica gel (200 g) 0-50% ethyl acetate/heptane to give the desired product as a cream powder (4 g, 7.2 mmol). ¹H NMR (CDCl₃) 1.31 3 Ht, 3.38 2 hs, 3.71 1 Hd, 4.10 1 Hd, 4.23 2 Hq, 4.55 2 Hd, 7.10-7.15 1 Hm, 7.45-7.52 1 Hm, 7.65-7.70 4 Hm. MH+555.

### Preparation 9: 3-(2-fluoro-5-(5-(3,4,5-trichlorophenyl)-5-(trifluoromethyl)-4,5-dihydroisoxazol-3-yl)benzylamino)-3-oxopropanoic acid

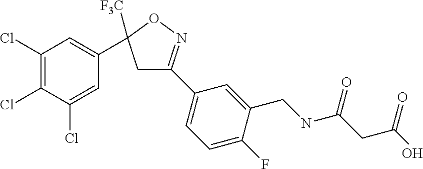

A slurry of 1-{2-fluoro-5-[5-(3,4,5-trichlorophenyl)-5-(trifluoromethyl)-4,5-dihydroisoxazol-3-yl]phenyl}methanamine hydrochloride (4 g, 7.2 mmol) in ethanol 40 mL was treated with 1N aqueous NaOH (40 mL) and stirred at room temperature for 2 hours. The reaction mixture was concentrated under reduced pressure to dryness and partitioned between ethyl acetate and 1N aqueous HCl. The organic extracts were dried and concentrated to give a white foam (3.7 g, 7.0 mmol, 97%) ¹H NMR (CDCl₃) 3.39 2Hs, 3.70 1 Hd, 4.08 1 Hd, 4.54 2 Hd 7.09-7.14 1 Hm, 7.38-7.41 1 Hm, 7.58-7.63 3 Hm, 7.66-7.68 1 Hm. MH+527.

### Example 1

### N-ethyl-N′-{2-fluoro-5-[5-(3,4,5-trichloro-phenyl)-5-trifluoromethyl-4,5-dihydro-isoxazol-3-yl]-benzyl}-malonamide

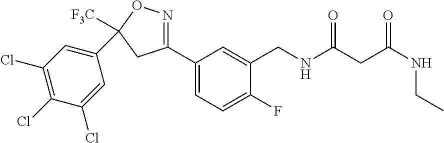

Ethylamine (2.44 mmol) was weighed into an 8 mL vial. A solution of 3-(2-fluoro-5-(5-(3,4,5-trichlorophenyl)-5-(trifluoromethyl)-4,5-dihydroisoxazol-3-yl)benzylamino)-3-oxopropanoic acid (0.082 mmol, 40 mg) in DMF 1 mL was added. A solution of O-(7-azabenzotriazol-1-yl)-N,N,N′,N′-tetramethyluronium hexafluorophosphate (0.09 mmol, 34.2 mg) in DMF (1 mL) was added, followed by triethylamine (0.82 mmol, 83 mg). The reaction mixture was shaken at ambient temperature for 72 hours. The reaction mixture was treated with MP-isocyanate resin (0.82 mmol, 560 mg, ˜1.47 mmol/g) and MP-carbonate resin (0.82 mmol, 260 mg, ˜3.14 mmol/g), and shaken at ambient temperature for 16 hours. The reaction was filtered and concentrated to give the crude product. The crude product was purified by preparative HPLC (Waters, Gemini NX C18 21×100 mm 5 μm, mobile phase A=0.1% trifluoroacetic acid in H₂O, mobile phase B=acetonitrile, linear gradient 30% B to 100% in 8 minutes, hold for 1 minute, 20 mL/minute, peaks collected by mass.) to give the desired product 16.3 mg. 29% yield MH+[554]. Retention time 2.84 minutes (Agilent 1200, Column=Gemini NX C18 4.6×50 mm 3 μm, mobile phase A=0.1% trifluoroacetic acid in H₂O, mobile phase B=acetonitrile, linear gradient 30% B to 100% in 5 minutes holding for 1 minute, 1.5 mL/minute).

### Example 2

### N-Cyclopropylmethyl-N′-{2-fluoro-5-[5-(3,4,5-trichloro-phenyl)-5-trifluoromethyl-4,5-dihydro-isoxazol-3-yl]-benzyl}-malonamide

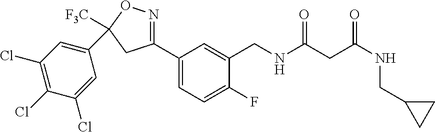

Cylopropanemethylamine (2.44 mmol) was weighed into an 8 mL vial. A solution of 3-(2-fluoro-5-(5-(3,4,5-trichlorophenyl)-5-(trifluoromethyl)-4,5-dihydroisoxazol-3-yl)benzylamino)-3-oxopropanoic acid (0.082 mmol, 40 mg) in DMF (1 mL) was added to the vial. A solution of 0-(7-azabenzotriazol-1-yl)-N,N,N′,N′-tetramethyluronium hexafluorophosphate (0.09 mmol, 34.2 mg) in DMF (1 ml) was added, followed by triethylamine (0.82 mmol, 83 mg). The reaction mixture was shaken at ambient temperature for 72 hours. The reaction mixture was treated with MP-isocyanate resin (0.82 mmol, 560 mg, ˜1.47 mmol/g) and MP-carbonate resin (0.82 mmol, 260 mg, ˜3.14 mmol/g), and shaken at ambient temperature for 16 hours. The reaction was filtered and concentrated under reduced pressure to give the crude product. The crude product was purified by preparative HPLC (Waters, Gemini NX C18 21×100 mm 5 μm, mobile phase A=0.1% trifluoroacetic acid in H₂O, mobile phase B=acetonitrile, linear gradient 30% B to 100% in 8 minutes, hold for 1 minute, 20 mL/min, peaks collected by mass.) to give the desired product 23.4 mg. 40% yield, MH+[580]. Retention time 3.09 minutes (Agilent 1200, Column=Gemini NX C18 4.6×50 mm 3 μm, mobile phase A=0.1% trifluoroacetic acid in H₂O, mobile phase B=acetonitrile, linear gradient 30% B to 100% in 5 minutes, holding for 1 minute, 1.5 mL/min).

### Example 3

### N-{5-[5-(3,4,5-trichlorophenyl)-5-(trifluoromethyl)-4,5-dihydroisoxazol-3-yl]-2-fluorobenzyl}acetamide

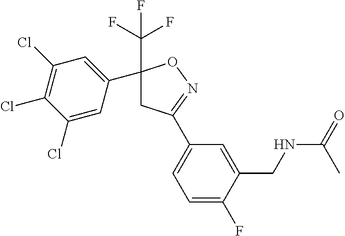

### Method A for Preparation of Amides

To a DMA (2 mL) mixture of 1-{2-fluoro-5-[5-(3,4,5-trichlorophenyl)-5-(trifluoromethyl)-4,5-dihydroisoxazol-3-yl]phenyl}methanamine hydrochloride (100 mg, 0.21 mmols) (Preparation 7) was added pyridine (72 mg, 0.9 mmol) followed by acetyl chloride (24 mg, 0.31 mmol). The reaction was allowed to stir for ten minutes at room temperature before water (25 mL) was added. The mixture was stirred for one hour at room temperature. The final product (95 mg, 94%) was collected by suction filtration as a white precipitate. ¹H NMR (400 MHz, CHLOROFORM-d) d ppm 2.05 (s, 3H) 3.69 (d, J=17.18 Hz, 1H) 4.09 (d, J=17.18 Hz, 1H) 4.50 (d, J=6.06 Hz, 2H) 5.90-6.00 (m, 1H) 7.08-7.17 (m, 1H) 7.62-7.70 (m, 4H) m/z (Cl) 483 [M+H]⁺.

### Example 4

### N-{2-fluoro-5-[5-(3,4,5-trichlorophenyl)-5-(trifluoromethyl)-4,5-dihydroisoxazol-3-yl]benzyl}-2-methylpropanamide

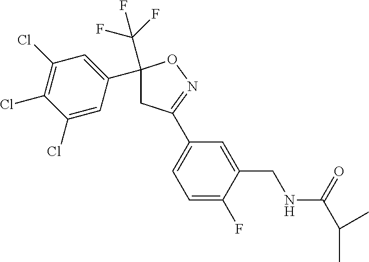

### Method B for Preparation of Amides

To a DMA (2 mL) solution of 1-{2-fluoro-5-[5-(3,4,5-trichlorophenyl)-5-(trifluoromethyl)-4,5-dihydroisoxazol-3-yl]phenyl}methanamine hydrochloride (45 mg, 0.094 mmol (Preparation 7) was added diisopropylethylamine (36.4 mg, 0.28 mmol), methylpropionic acid (12.5 mg, 0.14 mmol), EDC (23.4 mg, 0.12 mmol) and HOBT (1.2 mg, 0.009 mmol). The reaction was stirred at room temperature for 12 hours. The reaction mixture was partitioned between ethyl acetate (50 mL) and water (20 mL). The organic phase was washed with water (3×20 mL). Distillation of the solvent afforded the title compound, (42 mg, 87%), as a white solid. ¹H NMR (400 MHz, CDCl₃) δ ppm 1.19 (dd, J=6.82, 2.02 Hz, 6H) 2.41 (dt, J=13.83, 6.85 Hz, 1H) 3.68 (d, J=17.18 Hz, 1H) 4.08 (d, J=17.18 Hz, 1H) 4.51 (d, J=6.06 Hz, 2H) 5.89 (br. s., 1H) 7.06-7.20 (m, 1H) 7.59-7.71 (m, 4H); m/z (Cl) 511 [M+H]⁺.

### Example 5

### N-{2-fluoro-5-[5-(3,4,5-trichlorophenyl)-5-(trifluoromethyl)-4,5-dihydroisoxazol-3-yl]benzyl}cyclopropanecarboxamide

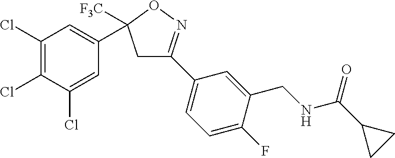

was prepared from 1-(2-fluoro-5-(5-(3,4,5-trichlorophenyl)-5-(trifluoromethyl)-4,5-dihydroisoxazol-3-yl)phenyl)methanamine (Preparation 7) through reaction with cyclopropanecarbonyl chloride according to Method A. ¹H NMR (400 MHz, CDCl₃) δ ppm 0.78-0.83 (m, 2H) 1.00-1.04 (m, 2H) 1.35-1.42 (m, 1H) 3.69 (d, J=17.43 Hz, 1H) 4.08 (d, J=17.18 Hz, 1H) 4.53 (d, J=6.32 Hz, 1H) 6.05 (br. s., 1H) 7.14 (t, J=8.97 Hz, 1H) 7.60-7.72 (m, 4H); m/z (Cl) 509 [M+H]⁺.

### Example 6

### N-{2-fluoro-5-[5-(3,4,5-trichlorophenyl)-5-(trifluoromethyl)-4,5-dihydroisoxazol-3-yl]benzyl}cyclobutanecarboxamide

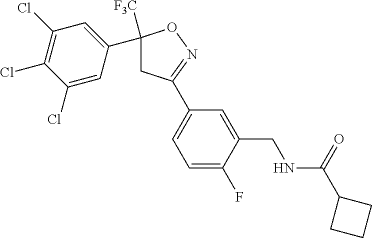

was prepared from 1-(2-fluoro-5-(5-(3,4,5-trichlorophenyl)-5-(trifluoromethyl)-4,5-dihydroisoxazol-3-yl)phenyl)methanamine hydrochloride (Preparation 7) through reaction with cyclobutanecarbonyl chloride according to Method A. ¹H NMR (400 MHz, CDCl₃) δ ppm 1.86-2.05 (m, 2H) 2.13-2.24 (m, 2H) 2.24-2.37 (m, 2H) 2.97-3.09 (m, 1H) 3.69 (d, J=16.93 Hz, 1H) 4.09 (d, J=17.18 Hz, 1H) 4.51 (d, J=6.32 Hz, 2H) 5.76 (br. s., 1H) 7.09-7.17 (m, 1H) 7.62-7.70 (m, 4H);); m/z (Cl) 525 [M+H]⁺.

### Example 7

### N-{2-fluoro-5-[5-(3,4,5-trichlorophenyl)-5-(trifluoromethyl)-4,5-dihydroisoxazol-3-yl]benzyl}propanamide

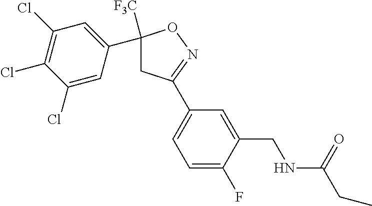

was prepared from 1-(2-fluoro-5-(5-(3,4,5-trichlorophenyl)-5-(trifluoromethyl)-4,5-dihydroisoxazol-3-yl)phenyl)methanamine hydrochloride through reaction with propionyl chloride according to Method A. ¹H NMR (400 MHz, CDCl₃) δ ppm 1.19 (t, J=7.58 Hz, 3H) 2.27 (q, J=7.58 Hz, 2H) 3.69 (d, J=17.18 Hz, 1H) 4.09 (d, J=17.18 Hz, 1H) 4.51 (d, J=6.32 Hz, 2H) 5.87 (br. s., 1H) 7.08-7.18 (m, 1H) 7.61-7.72 (m, 4H); m/z (Cl) 497 [M+H]⁺.

### Example 8

### 2-cyclopropyl-N-{2-fluoro-5-[5-(3,4,5-trichlorophenyl)-5-(trifluoromethyl)-4,5-dihydroisoxazol-3-yl]benzyl}acetamide

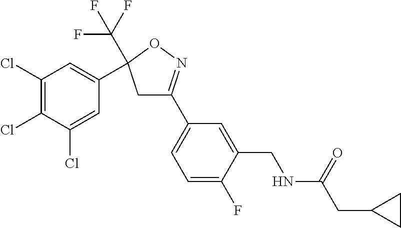

was prepared from 1-(2-fluoro-5-(5-(3,4,5-trichlorophenyl)-5-(trifluoromethyl)-4,5-dihydroisoxazol-3-yl)phenyl)methanamine hydrochloride through reaction with 2-cyclopropylacetic acid according to Method B. ¹H NMR (400 MHz, CDCl₃) δ ppm −0.07-0.07 (m, 2H) 0.36-0.48 (m, 2H) 0.76 (t, J=7.71 Hz, 1H) 1.98 (d, J=7.33 Hz, 2H) 3.45 (d, J=17.18 Hz, 1H) 3.85 (d, J=17.18 Hz, 1H) 4.31 (d, J=6.06 Hz, 2 H) 6.11 (br. s., 1H) 6.91 (t, J=9.47 Hz, 1H) 7.37-7.51 (m, 5H); m/z (Cl) 525 [M+H]⁺.

### Example 9

### N-{2-fluoro-5-[5-(3,4,5-trichlorophenyl)-5-(trifluoromethyl)-4,5-dihydroisoxazol-3-yl]benzyl}-3-methylbutanamide

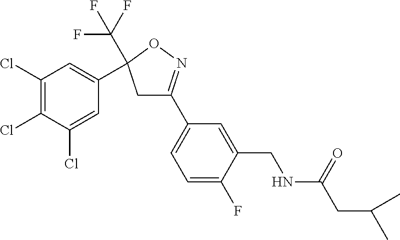

was prepared from (2-fluoro-5-(5-(3,4,5-trichlorophenyl)-5-(trifluoromethyl)-4,5-dihydroisoxazol-3-yl)phenyl)methanamine through reaction with 3-methylbutanoic acid according to Method B. ¹H NMR (400 MHz, CDCl₃) δ ppm 0.83-0.91 (m, 8 H) 1.01-1.14 (m, 1H) 1.95-2.13 (m, 4H) 3.59 (d, J=17.43 Hz, 1H) 3.99 (d, J=17.18 Hz, 1H) 4.42 (d, J=6.32 Hz, 2H) 5.79 (br. s., 1H) 6.97-7.11 (m, 1H) 7.48-7.65 (m, 4H); m/z (Cl) 527 [M+H]⁺.

### Preparation 10: tert-butyl 5-(5-(3,5-dichloro-4-fluorophenyl)-5-(trifluoromethyl)-4,5-dihydroisoxazol-3-yl)-2-fluorobenzylcarbamate

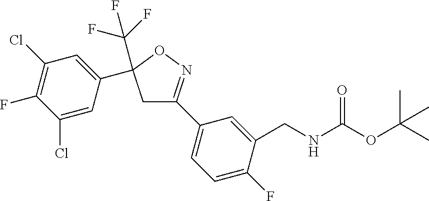

To a DMF (5 mL) solution of tert-butyl {2-fluoro-5-[(E/Z)-(hydroxyimino)methyl]benzyl}carbamate (Preparation 5, 250 mg, 0.826 mmols) was added n-chlorosuccinimide (115 mg, 0.858) in three equal portions over 30 minutes with approximately ten minutes between each addition. The reaction mixture was stirred in an atmosphere of nitrogen for twelve hours. To the crude reaction mixture was added 1,3-dichloro-2-fluoro-5-(1,1,1-trifluoroprop-2-en-2-yl)benzene (Preparation 2, 214 mg, 0.826 mmol) and solid sodium hydrogen carbonate (300 mg). The mixture was stirred at room temperature for 24 hours. The reaction mixture was partitioned between water (10 mL) and EtOAc (40 mL). The organic phase was washed successively with water (3×15 mL) dried (sodium sulfate), and the solvent distilled off at low pressure to give the crude product as a viscous colorless oil. The product was purified on silica gel (EtOAc gradient in hexanes) to afford the title compound as an amorphous glass (286 mg, 66%). m/z (Cl) 425 [M+H]⁺ (the Boc group is lost upon ionization) ¹H NMR (400 MHz, CHLOROFORM-d) d ppm 1.48 (s, 9H) 3.69 (d, J=17.43 Hz, 1H) 4.08 (d, J=17.18 Hz, 1H) 4.39 (d, J=6.06 Hz, 2H) 4.99 (br. s., 1H) 7.12 (t, J=9.09 Hz, 1H) 7.55-7.69 (m, 4H).

### Preparation 11. (5-(5-(3,5-dichloro-4-fluorophenyl)-5-(trifluoromethyl)-4,5-dihydroisoxazol-3-yl)-2-fluorophenyl)methanamine

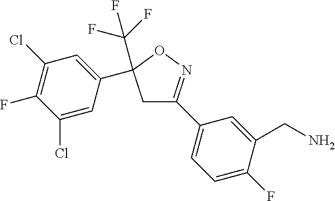

To a methylene chloride (10 mL) solution of tert-butyl 5-(5-(3,5-dichloro-4-fluorophenyl)-5-(trifluoromethyl)-4,5-dihydroisoxazol-3-yl)-2-fluorobenzylcarbamate (Preparation 10, 910 mg, 1.73 mmols)) was added TFA (2 mL). The reaction was stirred overnight at room temperature. The volatiles were distilled off at low pressure. The residual material was taken up in ethyl acetate (60 ml). The organic phase was washed with saturated aqueous sodium hydrogen carbonate solution (2×25 ml). The combined aqueous washes were back extracted with ethyl acetate (2×20 ml). The organic phases were all combined and dried over sodium sulfate. Distillation of solvent at low pressure afforded the product as a solid glass which was dried under vacuum. (736 mg, 99%) m/z (Cl) 425[M+H]⁺.

### Example 10

### 2-cyclopropyl-N-{5-[5-(3,5-dichloro-4-fluorophenyl)-5-(trifluoromethyl)-4,5-dihydroisoxazol-3-yl]-2-fluorobenzyl}acetamide

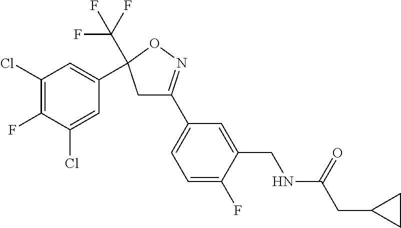

### Method C for Preparation of Amides

To a DMF (2 mL) solution of (5-(5-(3,5-dichloro-4-fluorophenyl)-5-(trifluoromethyl)-4,5-dihydroisoxazol-3-yl)-2-fluorophenyl)methanamine (48 mg, 0.118 mmol, Preparation 11) was added 2-cyclopropylacetic acid (15 mg, 0.15 mmol), triethylamine (55 mg, 0.542 mmol), HOBT (1.2 mg, 0.009 mmol) and HBTU (41.1 mg, 0.110 mmol). The reaction was stirred at room temperature for 12 hours. The reaction was filtered through a syringe filter. The filtrate was subjected to reverse phase HPLC purification to afford the final product (12 mg, 20%) as an amorphous glass. Retention time=3.33 minutes and m/z (Cl) 508.2 [M+H]⁺.

### Example 11

### N-{5-[5-(3,5-dichloro-4-fluorophenyl)-5-(trifluoromethyl)-4,5-dihydroisoxazol-3-yl]-2-fluorobenzyl}acetamide

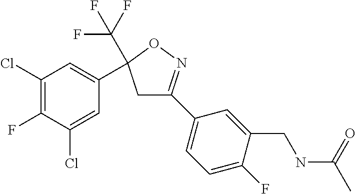

was prepared from (5-(5-(3,5-dichloro-4-fluorophenyl)-5-(trifluoromethyl)-4,5-dihydroisoxazol-3-yl)-2-fluorophenyl)methanamine through reaction with acetyl chloride according to Method A. ¹H NMR (400 MHz, CDCl₃) δ ppm 2.05 (s, 3H) 3.69 (d, J=17.43 Hz, 1H) 4.09 (d, J=17.18 Hz, 1H) 4.51 (d, J=6.06 Hz, 2H) 5.92 (br. s., 1H) 7.14 (t, J=8.97 Hz, 1H) 7.60 (d, J=6.06 Hz, 2H) 7.63-7.72 (m, 2H);); m/z (Cl) 467 [M+H]⁺.

### Example 12

### N-{5-[5-(3,5-dichloro-4-fluorophenyl)-5-(trifluoromethyl)-4,5-dihydroisoxazol-3-yl]-2-fluorobenzyl}cyclopropanecarboxamide

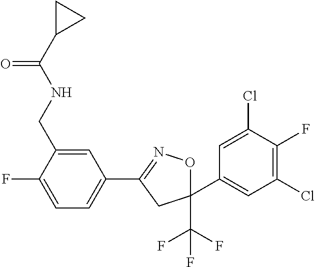

was prepared from (5-(5-(3,5-dichloro-4-fluorophenyl)-5-(trifluoromethyl)-4,5-dihydroisoxazol-3-yl)-2-fluorophenyl)methanamine through reaction with cyclopropane carboxylic in the presence of HBTU, HOBT and Hunig's base according to Method C. Analytical HPLC: Column=Waters X-Terra 3.5 μm 4.6×50 mm, mobile phase A=0.1% trifluoroacetic acid in H₂O, mobile phase B=acetonitrile, 50% B up to 100% B in 5 minutes, hold for 1 minute, 2 mL/minute. Retention Time: 3.94 minutes, m/z (Cl) 493.9 [M+H]⁺.

### Example 13

### N-{5-[5-(3,5-dichloro-4-fluorophenyl)-5-(trifluoromethyl)-4,5-dihydroisoxazol-3-yl]-2-fluorobenzyl}-3,3-difluorocyclobutanecarboxamide

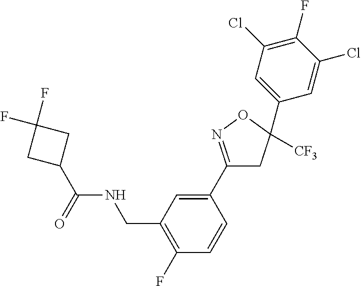

was prepared from (5-(5-(3,5-dichloro-4-fluorophenyl)-5-(trifluoromethyl)-4,5-dihydroisoxazol-3-yl)-2-fluorophenyl)methanamine through reaction with 3,3-difluorocyclobutane-carboxylic acid in the presence of HBTU, HOBT and Hunig's base according to Method C. Analytical HPLC: Column=Waters X-Terra 3.5 μm 4.6×50 mm, mobile phase A=0.1% trifluoroacetic acid in H₂O, mobile phase B=acetonitrile, 50% B up to 100% B in 5 minutes, hold for 1 minute, 2 mL/minute. Retention Time: 4.16 minutes, m/z (Cl) 543.9 [M+H]⁺.

### Preparation 12: methyl 5-bromo-2-chlorobenzoate

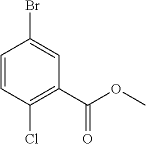

To a methylene chloride (50 ml) suspension of 2-chloro-5-bromobenzoic acid (10 g, 42 mmol) was added and excess of oxaly chloride and a drop of DMF. The reaction mixture stirred for twelve hours at room temperature in an atmosphere of nitrogen. All volatiles were removed by distillation at low pressure. The product, a viscous oil, was dissolved in methylene chloride (50 mL) and to the cooled solution (0° C.) was added methanol (5 mL). The solution was stirred for ten minutes at 0° C. and for one hour at room temperature. The volatiles were removed by distillation at low pressure to afford methyl 5-bromo-2-chlorobenzoate (10.5 g, 99%). ¹H NMR (400 MHz, CDCl₃) δ ppm 3.96 (s, 3H) 7.35 (d, J=8.59 Hz, 1H) 7.56 (dd, J=8.59, 2.27 Hz, 1H) 7.99 (d, J=2.53 Hz, 1H)

### Preparation 13. (5-bromo-2-chlorophenyl)methanol

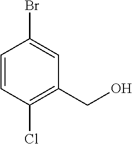

To a THF (50 mL) solution of methyl 5-bromo-2-chlorobenzoate (Preparation 12, 10.5 g, 42 mmol) was added sodium borohydride (3.18 g, 84 mmol) followed by the careful dropwise addition of MeOH (7 mL) over 30 minutes. The reaction was stirred for one hour at room temperature. An additional amount of sodium borohydride (0.5 g) was added and the mixture stirred for one more hour at room temperature. The reaction mixture was poured into ethyl acetate (125 mL) and stirred for twenty minutes. Water (50 mL) was added, slowly at first, then all at once. The layers were stirred vigorously together for fifteen minutes. The organic phase was collected, dried over sodium sulfate, and concentrated at low pressure to give (5-bromo-2-chlorophenyl)methanol as a white solid (7.85 g, 84%). ¹H NMR (400 MHz, CDCl₃) δ ppm 4.77 (s, 2H) 7.23 (d, J=8.34 Hz, 1H) 7.37 (dd, J=8.34, 2.53 Hz, 1H) 7.68 (d, J=2.53 Hz, 1H)

### Preparation 14. 5-bromo-2-chlorobenzyl methanesulfonate

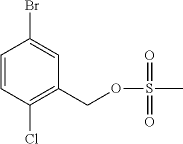

A methylene chloride (50 mL) solution of 5-bromo-2-chlorophenyl)methanol (Preparation 13, 7.85 g, 35.4 mmol) was cooled to 0° C. and methanesulfonyl chloride (4.06 g, 35.4 mmol) was added followed by the addition of triethylamine (3.64 g, 36 mmol). The solution was stirred at 0° C. for two hours and then for three hours at room temperature. Methyene chloride (50 mL) was added and the reaction mixture was washed with water. The organic phase was dried over sodium sulfate and concentrated at low pressure to give a colorless liquid that was purified on silica gel to provide the product, 5-bromo-2-chlorobenzyl methanesulfonate (7.62 g, 72%) ¹H NMR (400 MHz, CDCl₃) δ ppm 3.09 (s, 3H) 5.32 (s, 2H) 7.32 (d, J=8.59 Hz, 0H) 7.48 (d, J=2.27 Hz, 0H) 7.66 (d, J=2.27 Hz, 1H).

### Preparation 15. 2-(azidomethyl)-4-bromo-1-chlorobenzene

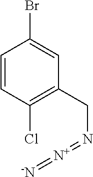

To a DMSO (30 mL) solution of 5-bromo-2-chlorobenzyl methanesulfonate (Preparation 14, 7.68 g, 25.6 mmol) was added sodium azide (1.75 g, 25.6 mmol). The reaction was stirred overnight at room temperature. Water (120 mL) was added to the reaction mixture. The product was extracted using EtOAc (2×100 mL). The combined extracts were then washed with water (6×50 mL). The organic phase was dried over sodium sulfate and the solvent distilled at low pressure and temperature (bath temp. below 40 C) to provide the product, 2-(azidomethyl)-4-bromo-1-chlorobenzene, as a glassy solid (5.78 g, 25.6 mmol). ¹H NMR (400 MHz, CDCl₃) δ ppm 4.48-4.53 (m, 1H) 7.25-7.34 (m, 1H) 7.43 (dd, J=8.59, 2.27 Hz, 1H) 7.58 (d, J=2.27 Hz, 2H)

### Preparation 16. 1-(5-bromo-2-chlorophenyl)methanamine hydrochloride

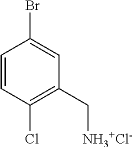

To a THF (70 mL) solution of 2-(azidomethyl)-4-bromo-1-chlorobenzene (Preparation 15) that had been cooled to 0° C. was added triphenylphosphine and water (6 mL). The reaction was stirred at 0° C. for one hour and then at room temperature for thirty six hours. The volatiles were removed by rotary evaporation at low pressure. The white residue was dissolved in EtOAc (70 mL). 4N HCl (6 mL) in dioxane was added and the mixture was stirred at 0° C. for two hours as the product precipitated out as the hydrochloride salt. The white precipitate was collected by suction filtration, washed with cold ethyl acetate (2×30 mL) and dried to give the 1-(5-bromo-2-chlorophenyl)methanamine hydrochloride (4.38 g, 73%). ¹H NMR (400 MHz, DMSO-d₆) δ ppm 4.12 (s, 2H) 7.51 (d, J=8.59 Hz, 1H) 7.63 (dd, J=8.59, 2.53 Hz, 1H) 7.89 (d, J=2.27 Hz, 1H) 8.61 (br. s., 3H).

### Preparation 17. tert-butyl (5-bromo-2-chlorobenzyl)carbamate

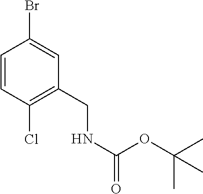

To a methylene chloride solution (70 mL) of 1-(5-bromo-2-chlorophenyl)methanamine hydrochloride (Preparation 16, 4.78 g, 18.6 mmol) that had been cooled to 0° C. was added Boc anhydride (4.06 g, 18.6 mmol) and triethylamine (4.14 g, 41 mmol). The reaction was stirred at room temperature for twenty four hours. The mixture was diluted with methylene chloride (40 mL) and washed with water (3×25 mL). The organic phase was dried over sodium sulfate and the solvent distilled off at low pressure. The crude liquid was purified on silica gel to provide tert-butyl (5-bromo-2-chlorobenzyl) carbamate (5.9 g, 17.5 mmol). ¹H NMR (400 MHz, CDCl₃) δ ppm 1.49 (s, 9H) 4.34-4.45 (m, 2H) 5.00 (br. s., 1 H) 7.24 (d, J=8.34 Hz, 1H) 7.35 (dd, 1H) 7.53 (d, J=2.53 Hz, 1H).

### Preparation 18. tert-butyl (2-chloro-5-formylbenzyl)carbamate

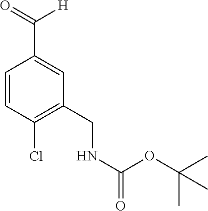

Tert-butyl (5-bromo-2-chlorobenzyl)carbamate (Preparation 17, 3.5 g, 10.9 mmol) was dissolved in anhydrous THF (50 mL). The solution was cooled to −78° C. in an atmosphere of nitrogen. n-BuLi (1.6 N in hexanes, 15 mL, 2.2 equiv)) was then added, dropwise, via addition funnel to the stirring mixture over fifteen minutes. The reaction was allowed to stir for ten more minutes at −78° C. in an atmosphere of nitrogen before DMF (2.41 g, 33 mmols) was added in a single aliquot. The cold bath was removed and the reaction was allowed to warm to room temperature over two hours. The reaction was then cooled to 0° C. and quenched by the addition of saturated aqueous ammonium chloride (50 mL). Water (100 mL) and EtOAc (200 mL) were then added and the layers mixed. The organic phase was collected, dried over sodium sulfate, and concentrated to a viscous oil. The crude oil was dissolved in CH₂Cl₂ (30 mL) and applied to an 80 g cartridge of silica gel. The cartridge was eluted with gradient of EtOAc in hexanes (5% to 60% over 6 column volumes) to give the pure product, tert-butyl (2-chloro-5-formylbenzyl)carbamate, (1.25 g, 42%), as thick amber oil. ¹H NMR (400 MHz, CDCl₃) δ ppm 1.49 (s, 9H) 4.50 (d, J=6.06 Hz, 2H) 5.10 (br. s., 1H) 7.55 (d, J=8.08 Hz, 1H) 7.76 (dd, J=8.08, 2.02 Hz, 1H) 7.91 (d, J=2.02 Hz, 1H) 10.01 (s, 1H).

### Preparation 19. tert-butyl {2-chloro-5-[(E/Z)-(hydroxylmino)methyl]benzyl}-carbamate

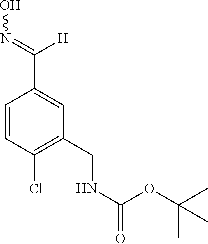

To an ethanolic (20 mL) solution of tert-butyl (2-chloro-5-formylbenzyl)-carbamate (Preparation 18, 1.25 g, 4.6 mmol) was added hydroxyl amine hydrochloride (0.95 g, 13.8 mmol) and sodium acetate (1.8 g, 23 mmol). The mixture was stirred for four hours at room temperature. The volatiles were removed by distillation at low pressure. The residual material was then partitioned between water (50 mL) and EtOAc (70 mL). The organic phase was dried (sodium sulfate) and concentrated to give the product, tert-butyl {2-chloro-5-[(E, Z)-(hydroxyimino)methyl]benzyl}carbamate (1.12 g, 85%).

### Preparation 20. tert-butyl {2-chloro-5-[(E/Z)-chloro(hydroxyimino)methyl]benzyl}-carbamate

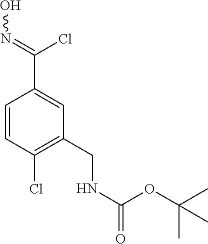

To a solution of tert-butyl {2-chloro-5-[(E/Z)-(hydroxyimino)methyl]benzyl}-carbamate (Preparation 19, 1.12 g, 3.9 mmol) in DMF (40 mL) was added N-chlorosuccinimide (0.525 g, 3.93 mmol). The solution was stirred for twelve hours at room temperature. The crude reaction mixture containing tert-butyl {2-chloro-5-[(E/Z)-(hydroxyimino)methyl]benzyl}carbamate was used directly in the next step.

### Preparation 21: tert-butyl 2-chloro-5-(5-(3,4,5-trichlorophenyl)-5-(trifluoromethyl)-4,5-dihydroisoxazol-3-yl)benzylcarbamate

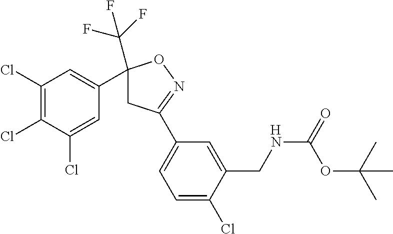

To a DMF (20 mL) solution of tert-butyl {2-chloro-5-[(E/Z)-chloro(hydroxyimino)-methylbenzyl}-carbamate (Preparation 20, 692 mg, 2.1 mmols) was added 1,2,3-trichloro-5-(1,1,1-trifluoroprop-2-en-2-yl)benzene (Preparation 1, 580 mg, 2.1 mmols) and solid sodium hydrogen carbonate (1000 mg). The mixture was stirred at room temperature for 24 hours. The reaction mixture was partitioned between water (10 mL) and EtOAc (40 mL). The organic phase was washed successively with water (3×15 mL) dried (sodium sulfate), and the solvent distilled off at low pressure to give the crude product as a viscous colorless oil. The product was purified on silica gel (EtOAc gradient in hexanes) to afford the title compound as an amorphous glass (890 mg, 76%). m/z (Cl) 459 [M+H]⁺ (the Boc group is lost upon ionization). ¹H NMR (400 MHz, CHLOROFORM-d) d ppm 1.48 (s, 9H) 3.69 (d, J=17.18 Hz, 1H) 4.08 (d, J=17.18 Hz, 1H) 4.44 (d, J=6.06 Hz, 2H) 5.02-5.12 (m, 1H) 7.44 (d, J=8.34 Hz, 1H) 7.53-7.68 (m, 4H).

### Preparation 22. 1-{2-chloro-5-[5-(3,4,5-trichlorophenyl)-5-(trifluoromethyl)-4,5-dihydroisoxazol-3-yl]phenyl}methanamine

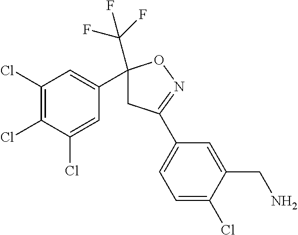

To a solution tert-butyl {2-chloro-5-[5-(3,4,5-trichlorophenyl)-5-(trifluoromethyl)-4,5-dihydroisoxazol-3-yl]benzyl}carbamate (Preparation 21, 920 mg, 1.65 mmol) in methylene chloride (10 mL) was added TFA (2 mL). The solution was stirred overnight at room temperature. The volatiles were removed by distillation at low pressure. The residue was taken up in ethyl acetate (60 mL). The organic phase was washed with saturated aqueous sodium hydrogen carbonate (2×25 mL). The combined aqueous washes were extracted with ethyl acetate (2×20 mL). All the organic extracts were combined and dried over sodium sulfate. The solvent was removed by distillation at low pressure to afford the product (595 mg, 79%). ¹H NMR (400 MHz, CDCl₃) δ ppm 3.68-3.74 (m, 1 H) 4.02 (s, 2H) 4.11 (d, J=17.18 Hz, 1H) 7.45 (d, J=8.34 Hz, 1H) 7.55 (dd, J=8.34, 2.02 Hz, 1H) 7.66 (s, 2H) 7.75 (d, J=2.02 Hz, 1H); m/z (Cl) 459 [M+H]⁺.

### Example 14

### N-{2-chloro-5-[5-(3,4,5-trichlorophenyl)-5-(trifluoromethyl)-4,5-dihydroisoxazol-3-yl]benzyl}acetamide

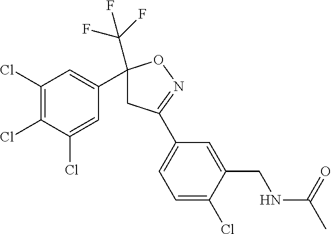

To a stirring mixture of 1-{2-chloro-5-[5-(3,4,5-trichlorophenyl)-5-(trifluoromethyl)-4,5-dihydroisoxazol-3-yl]phenyl}methanamine (Preparation 22, 50 mg, 0.11 mmol) and pyridine (0.1 mL) in DMF (3 mL) was added acetyl chloride (10 mg, 0.12 mmol). The reaction was stirred for ten minutes at room temperature. Water (12 mL) was added to precipitate the product. The white precipitate was collected using suction filtration. It was washed with water (6×10 mL) before being allowed to air dry overnight. The product, N-{2-chloro-5-[5-(3,4,5-trichlorophenyl)-5-(trifluoromethyl)-4,5-dihydroisoxazol-3-yl]benzyl}acetamide (40 mg, 73%) was obtained as a white solid. ¹H NMR (400 MHz, CDCl₃) δ ppm 2.06 (s, 3H) 3.69 (d, J=17.18 Hz, 1H) 4.09 (d, J=17.18 Hz, 1 H) 4.55 (d, J=6.32 Hz, 2H) 5.99 (br. s., 1H) 7.46 (d, J=8.34 Hz, 1H) 7.62 (dd, J=8.34, 2.02 Hz, 1H) 7.66 (s, 3H); m/z (Cl) 501 [M+H]⁺.

### Example 15

### N-{2-chloro-5-[5-(3,4,5-trichlorophenyl)-5-(trifluoromethyl)-4,5-dihydroisoxazol-3-yl]benzyl}cyclopropanecarboxamide

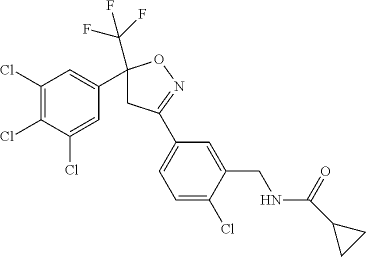

was made from 1-{2-chloro-5-[5-(3,4,5-trichlorophenyl)-5-(trifluoromethyl)-4,5-dihydroisoxazol-3-yl]phenyl}methanamine (Preparation 22) by reaction with cyclopropanecarbonyl chloride according to Method A. ¹H NMR (400 MHz, CDCl₃) δ ppm 0.78-0.83 (m, 2H) 0.99-1.03 (m, 2H) 1.37-1.45 (m, 1H) 3.69 (d, 17.43 Hz, 1H) 4.08 (d, J=17.43 Hz, 1H) 4.57 (d, J=6.32 Hz, 2H) 6.12-6.18 (m, 1H) 7.46 (d, J=8.34 Hz, 1H) 7.60-7.67 (m, 4H); m/z (Cl) 527 [M+H]⁺.

### Example 16

### N-{2-chloro-5-[5-(3,4,5-trichlorophenyl)-5-(trifluoromethyl)-4,5-dihydroisoxazol-3-yl]benzyl}-2-methylpropanamide

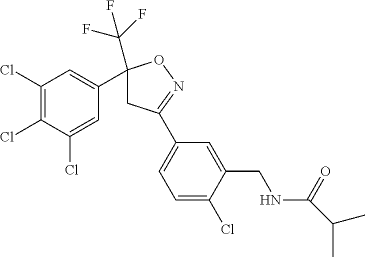

was made from 1-{2-chloro-5-[5-(3,4,5-trichlorophenyl)-5-(trifluoromethyl)-4,5-dihydroisoxazol-3-yl]phenyl}methanamine (Preparation 22) by reaction with isobutyryl chloride according to Method A ¹H NMR (400 MHz, CDCl₃) δ ppm 1.20 (dd, J=6.82, 2.27 Hz, 6H) 2.43 (s, 1H) 3.71 (s, 1H) 4.08 (d, J=17.18 Hz, 1H) 4.54 (d, J=6.06 Hz, 2H) 5.96-6.02 (m, 1H) 7.45 (d, J=8.34 Hz, 1H) 7.59-7.67 (m, 4H); m/z (Cl) 529 [M+H]⁺.

### Example 17

### N-{2-chloro-5-[5-(3,5-dichloro-4-fluorophenyl)-5-(trifluoromethyl)-4,5-dihydroisoxazol-3-yl]benzyl}acetamide

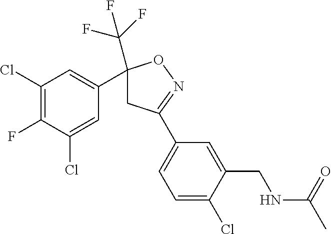

was made from (2-chloro-5-(5-(3,5-dichloro-4-fluorophenyl)-5-(trifluoromethyl)-4,5-dihydroisoxazol-3-yl)phenyl)methanamine by similar reaction with acetyl chloride according to Method A. ¹H NMR (400 MHz, CDCl₃) δ ppm 2.06 (s, 3H) 3.66-3.72 (m, 1H) 4.09 (d, J=17.18 Hz, 1H) 4.55 (d, J=6.32 Hz, 2H) 5.95-6.02 (m, 1 H) 7.46 (d, J=8.34 Hz, 1H) 7.58-7.64 (m, 3H) 7.65-7.67 (m, 1H); m/z (Cl) 485 [M+H]⁺.

### Preparation 23: methyl 4-bromo-3-(bromomethyl)benzoate

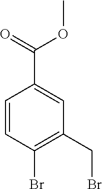

To a CCl₄ (30 mL) solution of 4-bromo-3-methyl benzoic acid methyl ester (10 g 43.6 mmol) was added NBS (4.8 g, 44 mmol) and a catalytic amount of benzolyl peroxide. The reaction was heated at reflux for eighteen hours. The mixture was cooled to room temperature and diluted with CH₂Cl₂ (50 mL). The organic phase was washed with water (3×20 mL) and concentrated using rotary evaporation at low pressure. The residual material was dissolved in hexanes and applied to a 120 g cartridge of silica gel. The product was eluted with an ethyl acetate gradient in hexanes to provide methyl 4-bromo-3-(bromomethyl)benzoate (7.73 g, 57%). ¹H NMR (400 MHz, CDCl₃) δ ppm 3.95 (s, 3H) 4.64 (s, 2H) 7.68 (d, J=8.34 Hz, 1H) 7.83 (dd, J=8.34, 2.02 Hz, 1H) 8.14 (d, J=2.02 Hz, 1H).

### Preparation 24. methyl 3-(azidomethyl)-4-bromobenzoate

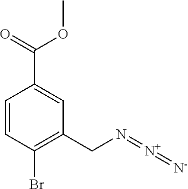

To a DMSO (40 mL) solution of methyl 4-bromo-3-(bromomethyl)benzoate (Preparation 23, 7.73 g, 25 mmol) was added sodium azide (1.63 g, 25 mmol).

The mixture was stirred at room temperature for four hours. The mixture was cooled in an ice bath and water (250 mL) was added to the reaction. A white precipitate appeared after stirring the mixture at 0° C. for one hour. The white solid was collected by suction filtration and washed with water to give methyl 3-(azidomethyl)-4-bromobenzoate (6.78 g, 100%)

### Preparation 25. methyl 3-(aminomethyl)-4-bromobenzoate

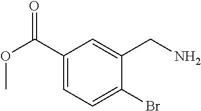

To a THF (70 mL) solution of methyl 3-(azidomethyl)-4-bromobenzoate (Preparation 24, 6.77 g, 25 mmol) was added water (6 mL) and triphenyl phosphine (6.57 g, 25.1 mmol). The mixture was stirred overnight at room temperature. The mixture was made acidic by the addition of 1 N HCl (aq) (40 mL). EtOAc (100 mL) and water (60 mL) were added. The layers were stirred vigorously together. The aqueous phase was collected and again washed with EtOAc (2×40 mL). The aqueous phase was then neutralized with saturated aqueous sodium hydrogen carbonate (40 mL). The product amine was then extracted with methylene chloride (3×40 mL). The combined extracts were dried over sodium sulfate and the solvent distilled off at low pressure to provide the methyl 3-(aminomethyl)-4-bromobenzoate (4.38 g, 72%). ¹H NMR (400 MHz, CDCl₃) δ ppm 3.94 (s, 3H) 3.99 (s, 2H) 7.65 (d, J=8.34 Hz, 1H) 7.79 (d, J=2.27 Hz, 1H) 8.09 (d, J=2.02 Hz, 1H).

### Preparation 26. methyl 4-bromo-3-((tert-butoxycarbonyl)methyl)benzoate

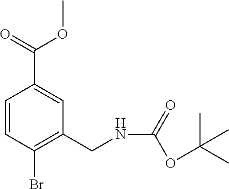

To a CH₂Cl₂ (25 mL) solution of 3-(aminomethyl)-4-bromobenzoate (Preparation 25, 4.38 g, 18 mmol) that had been cooled to 0° C. was added Boc anhydride (3.92 g, 18 mmol) followed by Hunig's base (2.58 g, 20 mmol). The reaction was stirred at 0° C. for one hour and then at room temperature for five hours. The volume was reduced to approximately 10 mL by distillation at low pressure. The residual liquid was appled to a cartridge of silica gel (80 g) and the cartridge was eluted with 25% EtOAc in Hexanes to provide methyl 4-bromo-3-((tert-butoxycarbonyl)methyl)benzoate (5.28 g, 85%) ¹H NMR (400 MHz, CDCl₃) 6 ppm 1.49 (s, 9H) 3.93 (s, 3H) 4.43 (d, 2H) 5.01-5.15 (m, 1H) 7.64 (d, J=8.34 Hz, 1H) 7.80 (d, J=2.02 Hz, 1H) 8.03 (s, 1H).

### Preparation 27. tert-butyl 2-bromo-5-(hydroxymethyl)benzylcarbamate

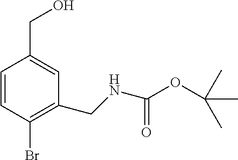

To a THF (50 mL) solution of 4-bromo-3-((tert-butoxycarbonyl)methyl)-benzoate (Preparation 26, 5.28 g, 15.3 mmol) was added sodium borohydride (579 mg, 15.3 mmol) in an atmosphere of nitrogen. To the stirring mixture, MeOH (10 mL) was added drop wise via addition funnel over twenty minutes. The reaction was warmed to 45° C. and stirred for one hour. A second equivalent of sodium borohydride (579 mg, 15.3 mmol) was added and stirring continued at 40° C. for two hours. The reaction was cooled to 0° C. and slowly quenched with saturated aqueous ammonium chloride. EtOAc (60 mL) and water (50 mL) were added. The layers were stirred vigorously together for fifteen minutes. The organic phase was collected, dried over sodium sulfate and the solvent distilled to provide tert-butyl 2-bromo-5-(hydroxymethyl)benzylcarbamate (4.74 g, 98%). ¹H NMR (400 MHz, CDCl₃) δ ppm 1.48 (s, 9H) 4.37-4.43 (m, 2H) 4.66-4.69 (m, 2 H) 5.01-5.09 (m, 1H) 7.16-7.20 (m, 1H) 7.38-7.40 (m, 1H) 7.55 (d, J=8.08 Hz, 1H).

### Preparation 28. tert-butyl 2-bromo-5-formylbenzylcarbamate

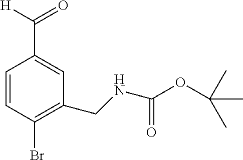

To a CH₂Cl₂ (50 mL) solution of tert-butyl 2-bromo-5-hydroxymethyl)benzyl-carbamate (Preparation 27, 4.73 g, 15 mmol) that had been cooled to 0° C. was added Dess-Martin periodinane (6.7 g, 15 mmol) in three portions over twenty minutes. The reaction mixture was allowed to warm to room temperature over two hours. The solvent was distilled off at low pressure. The residual material was dissolved in CH₂Cl₂ (100 mL), washed with saturated aqueous sodium hydrogen carbonate (3×40 mL). The organic phase was dried (sodium sulfate) and reduced volume with distillation at low pressure. The crude material was purified on silica gel to provide tert-butyl 2-bromo-5-formylbenzylcarbamate (1.2 g, 25%). ¹H NMR (400 MHz, CDCl₃) δ ppm 1.49 (s, 9H) 4.48 (d, J=6.32 Hz, 2H) 5.05-5.16 (m, 1H) 7.65-7.70 (m, 1H) 7.73-7.78 (m, 1H) 7.88 (d, J=2.02 Hz, 1 H) 10.01 (s, 1H).

### Preparation 29. tert-butyl 2-bromo-5-((hydroxyimino)methyl)benzylcarbamate

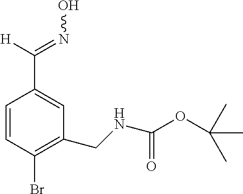

To an ethanolic (20 mL) solution of tert-butyl 2-bromo-5-formylbenzylcarbamate (Preparation 28, 1.15 g, 3.7 mmol) was added hydroxyl amine hydrochloride (260 mg, 3.8 mmol) and sodium acetate (5 equiv). The mixture was stirred for four hours at room temperature. The volatiles were distilled of at low pressure. The residual material was partitioned between water (50 mL) and EtOAc (70 mL). The organic phase was dried (sodium sulfate) and concentrated to give tert-butyl 2-bromo-5-((hydroxyimino)methyl)benzylcarbamate (1.18 g, 98%)

### Preparation 30. tert-butyl 2-bromo-5-(chloro(hydroxyimino)methyl)benzyl-carbamate

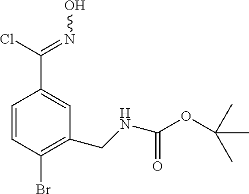

To a DMF (40 mL) solution of tert-butyl 2-bromo-5-((hydroxyimino)methyl)benzylcarbamate (Preparation 29, 1.18 g, 3.6 mmol) was added N-chlorosuccinimide (0.48 g, 3.6 mmol). The solution was stirred for twelve hours at room temperature. The crude reaction mixture containing tert-butyl 2-bromo-5-(chloro(hydroxyimino)methyl)benzylcarbamate was used directly in the next step.

### Preparation 31. tert-butyl 2-bromo-5-(5-(3,4,5-trichlorophenyl)-5-(trifluoromethyl)-4,5-dihydroisoxazol-3-yl)benzylcarbamate

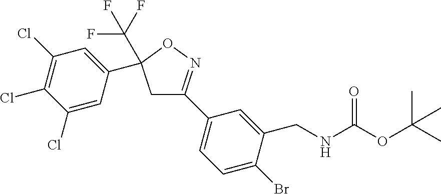

To a solution (20 mL) of tert-butyl 2-bromo-5-(chloro(hydroxyimino)-methyl)benzylcarbamate (Preparation 26, 700 mg, 1.9 mmol) was added 1,2,3-trichloro-5-(1,1,1-trifluoroprop-2-en-2-yl)benzene (Preparation 1, 530 mg, 1.92 mmol) and sodium hydrogen carbonate (1 g). The mixture stirred at room temperature for twelve hours. Reaction mixture was partitioned between water (100 mL) and ethyl ether (120 mL). The organic phase was dried over sodium sulfate and the solvent was distilled off. The residual oil was purified on silica gel using EtOAc/hexanes as the mobile phase to provide tert-butyl 2-bromo-5-(5-(3,4,5-trichlorophenyl)-5-(trifluoromethyl)-4,5-dihydroisoxazol-3-yl)benzyl-carbamate (854 mg, 60%).

### Preparation 32. (2-bromo-5-(5-(3,4,5-trichlorophenyl)-5-(trifluoromethyl)-4,5-dihydroisoxazol-3-yl)phenyl)methanamine

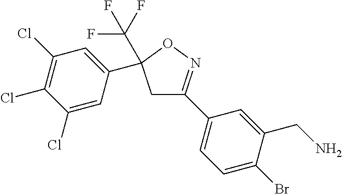

To a solution of tert-butyl 2-bromo-5-(5-(3,4,5-trichlorophenyl)-5-(trifluoromethyl)-4,5-dihydroisoxazol-3-yl)benzylcarbamate (Preparation 31, 844 mg, 1.4 mmol) in CH₂Cl₂ (10 mL) was added TFA (2 mL). The solution was stirred overnight at room temperature. The volatiles were removed by distillation at low pressure. The residue was taken up in ethyl acetate (60 mL). The organic phase was washed with saturated aqueous sodium hydrogen carbonate (2×25 mL). The combined aqueous washes were extracted with ethyl acetate (2×20 mL). All the organic extracts were combined and dried over sodium sulfate. The solvent was removed by distillation at low pressure to provide the product (2-bromo-5-(5-(3,4,5-trichlorophenyl)-5-(trifluoromethyl)-4,5-dihydroisoxazol-3-yl)phenyl)methanamine (677 mg, 1.4 mmol). m/z (Cl) 503 [M+H]⁺.

### Example 18

### N-(2-bromo-5-(5-(3,4,5-trichlorophenyl)-5-(trifluoromethyl)-4,5-dihydroisoxazol-3-yl)benzyl)acetamide

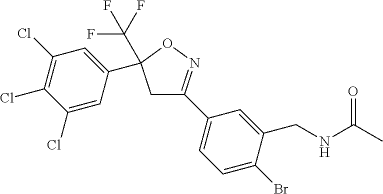

To a stirring mixture of (2-bromo-5-(5-(3,4,5-trichlorophenyl)-5-(trifluoromethyl)-4,5-dihydroisoxazol-3-yl)phenyl)methanamine (Preparation 32, 70 mg, 0.14 mmol) and pyridine (0.1 mL) in DMF (3 mL) was added acetyl chloride (11 mg, 0.14 mmol). The reaction was stirred for ten minutes at room temperature. Water (12 mL) was added to precipitate the product. The white precipitate was collected using suction filtration. It was washed with water (6×10 ml) before being allowed to air dry overnight. The product N-(2-bromo-5-(5-(3,4,5-trichlorophenyl)-5-(trifluoromethyl)-4,5-dihydroisoxazol-3-yl)benzyl)acetamide (55 mg, 73%) was obtained as a white solid. ¹H NMR (400 MHz, CDCl₃) δ ppm 2.06 (s, 3H) 3.66-3.74 (m, J=17.18 Hz, 1H) 4.09 (d, J=17.18 Hz, 1H) 4.54 (d, J=6.32 Hz, 2H) 5.98-6.05 (m, 1H) 7.51-7.56 (m, 1H) 7.62-7.67 (m, 4H);). m/z (Cl) 545 [M+H]⁺.

### Biological Assays

The biological activity of the compounds of the present invention were tested against hard tick larvae, soft ticks, horn flies, and fleas, using the test methods described below.

Hard Tick Larvae (Rhipicephalus sanquineus) Whole Organism Contact Assay

Formula (1) compounds were dissolved in isopropyl alcohol (IPA) and aliquots were added to vials placed on a roller for at least 2 hours to allow the IPA to evaporate. IPA alone was used as a negative control and fipronil was used as a positive control. Approximately 50-200 tick larvae were added to the vials using a swab and the vials were closed. At approximately 24 and 48 hours, the vials were examined and knockdown was recorded as active. Vials showing knockdown were examined for tick paralysis and/or death at approximately 48 hrs. Endpoint data can be recorded as an effective dose 100% (ED¹⁰⁰) and/or a lethal dose 100% (LD¹⁰⁰) in μg/cm². Examples 1 and 2 demonstrated an ED¹⁰⁰ of 10.0 μg/cm². Examples 3-9, 11, and 14-18 demonstrated an ED¹⁰⁰ of ≦1.0 μg/cm², and wherein Examples 5-7 and 11 demonstrated an ED¹⁰⁰ of ≦0.1 μg/cm².

Soft Tick (Ornithidorus turicata) Blood Feed Assay

Formula (1) compounds were dissolved in dimethylsulfoxide (DMSO) and aliquots were added to citrated bovine blood in a membrane covered Petri dish. The Petri dish was then placed on a warming tray. Approximately 5 nymph stage ticks were placed onto the membrane, covered, and left to feed. Fed ticks were removed and placed into a Petri dish with sand. Fed ticks were observed at approximately 24, 48 and 72 hours for paralysis and/or death. Endpoint data can be recorded as an ED¹⁰⁰ and/or an LD¹⁰⁰ in μg/mL. Positive control was fipronil and DMSO was used for the negative control. In this assay, Examples 3 and 11 demonstrated an ED¹⁰⁰ of ≦1 μg/cm².

Horn Fly (Haematobia irritans) Feed Assay

Formula (1) compounds were dissolved in DMSO and aliquots were added to citrated bovine blood in a membrane covered Petri dish. Approximately ten horn flies were placed onto each Petri dish and covered. The flies were allowed to feed on the treated blood cell. Flies were held at approximately 80° F. with a minimum of approximately 50% relative humidity. Flies were examined for knockdown and mortality at approximately 2 and 24 hours. Endpoint data were recorded as a lethal dose 90% (LD⁹⁰) in μg/mL. In this assay, Example 3 demonstrated an LD⁹⁰ of 10 μg/mL. In this assay, Examples 6, 8, 9, and 11 demonstrated an LD⁹⁰ of 3 μg/mL. Further, in this assay, Examples 4, 5, 7, 17, and 18 demonstrated an LD⁹⁰ of 1 μg/mL.

Flea (Ctenocephalides felis) Membrane Feed Assay-Adult

Formula (1) compounds were dissolved in DMSO and aliquots were added to citrated bovine blood in a membrane covered Petri dish pre-warmed to 37° C. Feeding tubes containing approximately 30-35 adult fleas were placed onto the Petri dishes. The fleas were allowed to feed for approximately 2 hours. Fleas were observed for knockdown and/or death at approximately 2 and 24 hours. Endpoint data were recorded as an efficacious dose 80% (ED⁸⁰) in μg/mL. In this assay, Examples 6, 7, and 18 demonstrated an ED⁸⁰ of 10 μg/mL. Further, in this assay, Examples 3, 5, 12, and 13 demonstrated an ED⁸⁰ of 3 μg/mL.

We claim:

1. A compound of Formula (1) 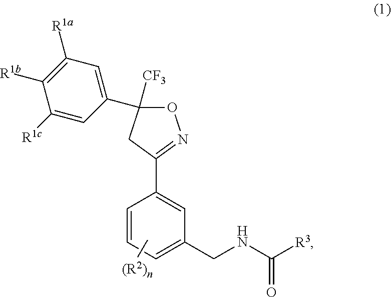
or a veterinarily acceptable salt thereof, wherein
R^{1a}, R^{1b}, and R^{1c} are each independently selected from halogen, cyano, C₁-C₈ alkyl, C₁-C₆ haloalkyl, and C₁-C₆ haloalkoxy, and each R¹ may be identical with or different from each other;
R² is hydrogen, halo, cyano, C₁-C₆ alkyl, C₁-C₆ haloalkyl, or C₁-C₆ alkoxy, C₁-C₆ haloalkoxy, C₃-C₆ cycloalkyl, where n is an integer 1, 2, or 3, and when n is 2 or 3, each R² may be identical with or different from each other;
R³ is selected from C₁-C₈ alkyl, C₀-C₃ alkylC₃-C₆ cycloalkyl, C₁-C₆ alkyl-OR⁴, or C₁-C₆ alkylC(O)NR^{a}R^{b}, wherein the C₁-C₈ alkyl and the C₀-C₃ alkylC₃-C₆ cycloalkyl are optionally substituted with at least one substituent selected from halo, cyano, hydroxyl, and S(O)ₚR⁴;
R⁴ is C₁-C₆ alkyl or C₁-C₆ haloalkyl;
R^{a} is hydrogen or C₁-C₆ alkyl;
R^{b} is hydrogen, C₁-C₆ alkyl, C₁-C₆ haloalkyl, C₀-C₄alkylC₃-C₆cycloalkyl, or C₁-C₃alkylHet, wherein Het is a 5- or 6-membered monocyclic aromatic ring containing at least one heteroatom selected from N, O, or S, and the Het can be optionally substituted with at least one substituent selected from halo, cyano, C₁-C₆ alkyl, and C₁-C₆ haloalkyl; and
p is the integer 0, 1, or 2.

2. The compound of claim 1 wherein
R^{1a}, R^{1b}, and R^{1c} are each independently selected from halogen, cyano, C₁-C₈ alkyl, and C₁-C₆ haloalkyl, or one of R^{1a}, R^{1b} or R^{1c} is SO₂CF₃.

3. The compound of claim 2 having Formula (1A), (1B), (1C), or (1D) 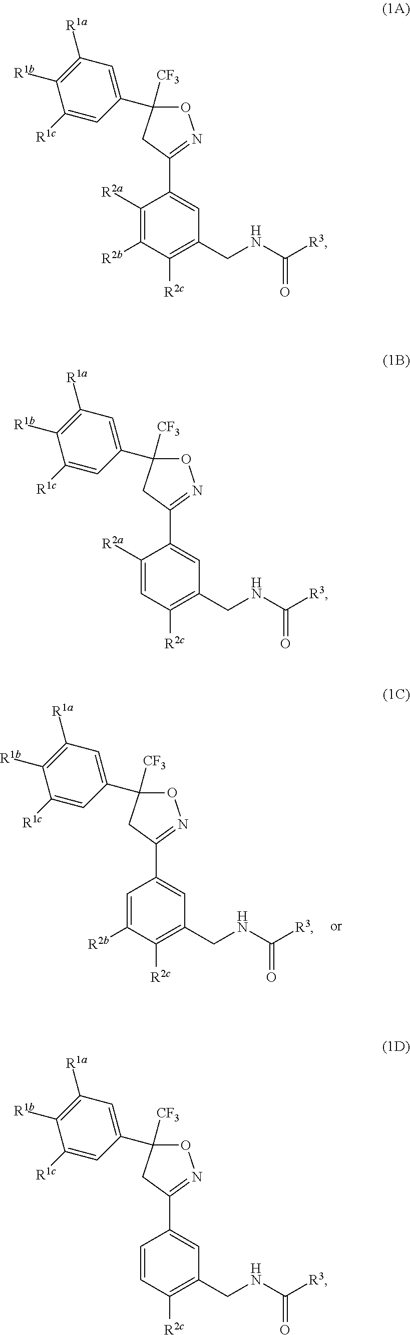
or a veterinarily acceptable salt thereof, wherein
R^{1a}, R^{1b}, and R^{1c} are each independently selected from halogen, cyano, C₁-C₈ alkyl, and C₁-C₆ haloalkyl, or one of R^{1a}, R^{1b}, or R^{1c} is SO₂CF₃, and
R^{2a}, R^{2b}, and R^{2c} are each independently hydrogen, halo, cyano, C₁-C₆ alkyl, C₁-C₆ haloalkyl, or C₃-C₆cycloalkyl.

4. The compound of claim 3 having Formula (1D) 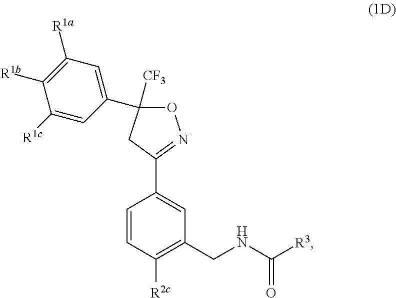
or a veterinarily acceptable salt thereof, wherein
R^{1a}, R^{1b}, and R^{1c} are each independently selected from halogen, cyano, C₁-C₈alkyl, and C₁-C₆ haloalkyl, or one of R^{1a}, R^{1b}, or R^{1c} is —SO₂CF₃; and
R^{2c} is hydrogen, halo, cyano, methyl, ethyl, —CF₃, —CH₂CF₃, cyclopropyl or cyclobutyl.

5. The compound of claim 4 wherein
R^{1a}, R^{1b}, and R^{1c} are each independently selected from fluoro, chloro, bromo, cyano, methyl, ethyl, —CF₃, and —CH₂CF₃; and
R^{2c} is hydrogen, fluoro, chloro, bromo, cyano, methyl, or CF₃.

6. The compound of claim 5 wherein R^{1a}, R^{1b}, and R^{1c} are each independently selected from fluoro, chloro, bromo, and CF₃; and
R^{2c} is fluoro, chloro, bromo, methyl, or CF₃.

7. The compound of claim 6 wherein
R³ is selected from C₁-C₈ alkyl or C₀-C₃ alkylC₃-C₆ cycloalkyl; wherein the C₁-C₈ alkyl and the C₀-C₃ alkylC₃-C₆ cycloalkyl are optionally substituted with at least one substituent selected from halo, hydroxyl, and S(O)ₚR⁴ where p is the integer 0, 1, or 2, and R⁴ is methyl, ethyl, or isopropyl.

8. The compound of claim 7 wherein
R³ is selected from C₁-C₈ alkyl, cyclopropyl, cyclobutyl, cyclopentyl, methylcyclopropyl, ethylcyclopropyl, methylcyclobutyl, ethylcyclobutyl, and methyl cyclopentyl; wherein the C₁-C₈ alkyl and the cycloalkyl or alkyl cycloalkyl are optionally substituted with at least one substituent selected from halo, hydroxyl, —SCH₃, and —S(O)₂CH₃.

9. A compound of claim 1 selected from
N-{5-[5-(3,4,5-trichlorophenyl)-5-(trifluoromethyl)-4,5-dihydroisoxazol-3-yl]-2-fluorobenzyl}acetamide;
N-{2-fluoro-5-[5-(3,4,5-trichlorophenyl)-5-(trifluoromethyl)-4,5-dihydroisoxazol-3-yl]benzyl}-2-methylpropanamide;
N-{2-fluoro-5-[5-(3,4,5-trichlorophenyl)-5-(trifluoromethyl)-4,5-dihydroisoxazol-3-yl]benzyl}cyclopropanecarboxamide;
N-{2-fluoro-5-[5-(3,4,5-trichlorophenyl)-5-(trifluoromethyl)-4,5-dihydroisoxazol-3-yl]benzyl}cyclobutanecarboxamide;
N-{2-fluoro-5-[5-(3,4,5-trichlorophenyl)-5-(trifluoromethyl)-4,5-dihydroisoxazol-3-yl]benzyl}propanamide;
2-cyclopropyl-N-{2-fluoro-5-[5-(3,4,5-trichlorophenyl)-5-(trifluoromethyl)-4,5-dihydroisoxazol-3-yl]benzyl}acetamide;
N-{2-fluoro-5-[5-(3,4,5-trichlorophenyl)-5-(trifluoromethyl)-4,5-dihydroisoxazol-3-yl]benzyl}-3-methylbutanamide;
2-cyclopropyl-N-{5-[5-(3,5-dichloro-4-fluorophenyl)-5-(trifluoromethyl)-4,5-dihydroisoxazol-3-yl]-2-fluorobenzyl}acetamide;
N-{5-[5-(3,5-dichloro-4-fluorophenyl)-5-(trifluoromethyl)-4,5-dihydroisoxazol-3-yl]-2-fluorobenzyl}acetamide;
N-{5-[5-(3,5-dichloro-4-fluorophenyl)-5-(trifluoromethyl)-4,5-dihydroisoxazol-3-yl]-2-fluorobenzyl}cyclopropanecarboxamide;
N-{5-[5-(3,5-dichloro-4-fluorophenyl)-5-(trifluoromethyl)-4,5-dihydroisoxazol-3-yl]-2-fluorobenzyl}-3,3-difluorocyclobutanecarboxamide
N-{2-chloro-5-[5-(3,4,5-trichlorophenyl)-5-(trifluoromethyl)-4,5-dihydroisoxazol-3-yl]benzyl}acetamide;
N-{2-chloro-5-[5-(3,4,5-trichlorophenyl)-5-(trifluoromethyl)-4,5-dihydroisoxazol-3-yl]benzyl}cyclopropanecarboxamide;
N-{2-chloro-5-[5-(3,4,5-trichlorophenyl)-5-(trifluoromethyl)-4,5-dihydroisoxazol-3-yl]benzyl}-2-methylpropanamide;
N-{2-chloro-5-[5-(3,5-dichloro-4-fluorophenyl)-5-(trifluoromethyl)-4,5-dihydroisoxazol-3-yl]benzyl}acetamide;
N-(2-bromo-5-(5-(3,4,5-trichlorophenyl)-5-(trifluoromethyl)-4,5-dihydroisoxazol-3-yl)benzyl)acetamide;
N-Cyclopropylmethyl-N′-{2-fluoro-5-[5-(3,4,5-trichloro-phenyl)-5-trifluoromethyl-4,5-dihydro-isoxazol-3-yl]-benzyl}-malonamide; and
N-ethyl-N′-{2-fluoro-5-[5-(3,4,5-trichloro-phenyl)-5-trifluoromethyl-4,5-dihydro-isoxazol-3-yl]benzyl}malonamide;
or a veterinarily acceptable salt thereof.

10. A veterinary composition comprising a therapeutically effective amount of a compound of Formula (1) 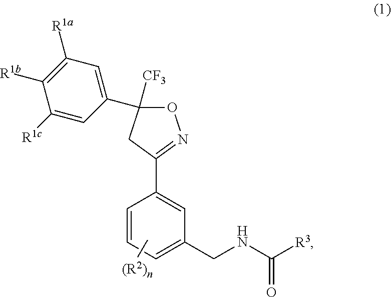
or a veterinarily acceptable salt thereof, wherein
R^{1a}, R^{1b}, and R^{1c} are each independently selected from halogen, cyano, C₁-C₈ alkyl, C₁-C₆ haloalkyl, and C₁-C₆ haloalkoxy, and each R¹ may be identical with or different from each other;
R² is hydrogen, halo, cyano, C₁-C₆ alkyl, C₁-C₆ haloalkyl, or C₁-C₆ alkoxy, C₁-C₆ haloalkoxy, C₃-C₆cycloalkyl, where n is an integer 1, 2, or 3, and when n is 2 or 3, each R² may be identical with or different from each other;
R³ is selected from C₁-C₈ alkyl, C₀-C₃ alkylC₃-C₆ cycloalkyl, C₁-C₆ alkyl-OR⁴, or C₁-C₆ alkylC(O)NR^{a}R^{b}, wherein the C₁-C₈ alkyl and the C₀-C₃ alkylC₃-C₆ cycloalkyl are optionally substituted with at least one substituent selected from halo, cyano, hydroxyl, and S(O)ₚR⁴;
R⁴ is C₁-C₆ alkyl or C₁-C₆ haloalkyl;
R^{a} is hydrogen or C₁-C₆ alkyl;
R^{b} is hydrogen, C₁-C₆ alkyl, C₁-C₆ haloalkyl, C₀-C₄alkylC₃-C₆cycloalkyl, or C₁-C₃alkylHet, wherein Het is a 5- or 6-membered monocyclic aromatic ring containing at least one heteroatom selected from N, O, or S, and the Het can be optionally substituted with at least one substituent selected from halo, cyano, C₁-C₆ alkyl, and C₁-C₆ haloalkyl; and
p is the integer 0, 1, or 2.

11. The veterinary composition of claim 10 further comprising a veterinarily acceptable excipient, diluent, or carrier.

12. The veterinary composition of claim 11 further comprising at least one additional veterinary agent.

13. A method for the treatment of parasites in an animal or bird comprising administering to said animal or bird an effective amount of a compound of Formula (1) 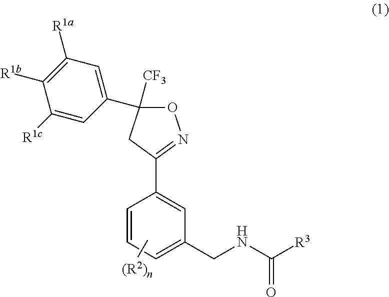
or a veterinarily acceptable salt thereof, wherein
R^{1a}, R^{1b}, and R^{1b} are each independently selected from halogen, cyano, C₁-C₈ alkyl, C₁-C₆ haloalkyl, and C₁-C₆ haloalkoxy, and each R¹ may be identical with or different from each other;
R² is hydrogen, halo, cyano, C₁-C₆ alkyl, C₁-C₆ haloalkyl, or C₁-C₆ alkoxy, C₁-C₆ haloalkoxy, C₃-C₆cycloalkyl, where n is an integer 1, 2, or 3, and when n is 2 or 3, each R² may be identical with or different from each other;
R³ is selected from C₁-C₈ alkyl, C₀-C₃ alkylC₃-C₆ cycloalkyl, C₁-C₆ alkyl-OR⁴, or C₁-C₆ alkylC(O)NR^{a}R^{b}, wherein the C₁-C₈ alkyl and the C₀-C₃ alkylC₃-C₆ cycloalkyl are optionally substituted with at least one substituent selected from halo, cyano, hydroxyl, and S(O)ₚR⁴;
R⁴ is C₁-C₆ alkyl or C₁-C₆ haloalkyl;
R^{a} is hydrogen or C₁-C₆ alkyl;
R^{b} is hydrogen, C₁-C₆ alkyl, C₁-C₆ haloalkyl, C₀-C₄alkylC₃-C₆cycloalkyl, or C₁-C₃alkylHet, wherein Het is a 5- or 6-membered monocyclic aromatic ring containing at least one heteroatom selected from N, O, or S, and the Het can be optionally substituted with at least one substituent selected from halo, cyano, C₁-C₆ alkyl, and C₁-C₆ haloalkyl; and
p is the integer 0, 1, or 2.

14. The method of claim 13 wherein the compound is administered orally or topically and the animal is a companion animal or livestock and the bird is fowl.

15. The method of claim 14 wherein the companion animal is dog, cat, and horse, and livestock is bovine and ovine.
